(19) United States
(12) Patent Application Publication
(10) Pub. No.: US 2022/0084630 A1
(43) Pub. Date: Mar. 17, 2022

(54) METHODS AND KITS FOR DETECTING PATHOGENS

(71) Applicant: Clear Labs, Inc., San Carlos, CA (US)

(72) Inventors: Sasan AMINI, San Carlos, CA (US); Ramin KHAKSAR, San Carlos, CA (US); Michael TAYLOR, San Carlos, CA (US); Julius Christopher BARSI, San Carlos, CA (US); Hossein NAMAZI, San Carlos, CA (US); Adam ALLRED, San Carlos, CA (US); Shaokang ZHANG, San Carlos, CA (US); Henrik GEHRMANN, San Carlos, CA (US); Kyle S. RHODEN, San Carlos, CA (US); Shadi SHOKRALLA, San Carlos, CA (US); Daniel MCDONOUGH, San Carlos, CA (US); Prasanna Thwar KRISHNAN, San Carlos, CA (US)

(21) Appl. No.: 17/534,000

(22) Filed: Nov. 23, 2021

Related U.S. Application Data
(63) Continuation of application No. PCT/US20/34329, filed on May 22, 2020.
(60) Provisional application No. 62/852,794, filed on May 24, 2019.
(60) Provisional application No. 62/878,238, filed on Jul. 24, 2019.

(51) Int. Cl. G16B 30/10; G16B 10/00

(52) U.S. Cl. CPC G16B 30/10; G16B 10/00

(57) ABSTRACT

Food processing facilities should employ environmental sampling programs to monitor for general levels of hygiene (the efficacy of general cleaning and sanitation for the removal of transient microorganisms). The instant disclosure provides kits, systems and methods for amplifying a portion of a genome of a pathogen at a plurality of physical locations within a facility; and associating, via a computer, the presence of said pathogen with a location of the plurality of physical locations within said facility.

### CROSS-REFERENCE

This application is a continuation of PCT Application No. PCT/US20/34329, filed May 22, 2020; which claims the benefit of U.S. Provisional Application No. 62/852,794, filed on May 24, 2019, and U.S. Provisional Application No. 62/878,238, filed on Jul. 24, 2019; each of which is incorporated herein in its entireties.

### BACKGROUND

Microorganisms are typically present in food handling environments. These microorganisms can be characterized as belonging to two distinct groups: transient and resident. Transient microorganisms are usually introduced into the food environment through raw materials, water and employees. Normally the routine application of good sanitation practices is able to kill these organisms. However, if contamination levels are high or sanitation procedures are inadequate, transient microorganisms may be able to establish themselves, multiply and become resident. Organisms such as Coliforms and Salmonella spp. and Listeria spp. have a well-established history of becoming residents in food handling environments, as well as other high traffic environments such as medical facilities.

### SUMMARY

In some aspects, the disclosure provides an environmental sampling program that monitors the presence of specific pathogens that may be present as transient or resident microorganisms. The detection of specific pathogens serves two important roles. Firstly, it highlights the presence of important food pathogens which may have been introduced into a food handling or medical environment but may not have been eliminated by routine sanitation practices and therefore may be passed onto food or medical materials. Secondly, it assists in determining sources of these important pathogens that may be resident.

A pathogen detection system (such as a deployable system) may be designed to assay samples from multiple environments, including that can, e.g. a food processing facility, a hospital, a pharmacy, or any type of medical or clinical facility. In most cases, it is highly desirable to have a device that is highly automated to reduce the number of steps that a user must be involved in to increase the ease of usage and reduce the risk of contamination or other sources of process failure.

In some aspects the disclosure provides for a kit comprising: (a) reagents for performing a PCR amplification reaction on a food or environmental sample from a food processing facility for detecting a Listeria monocytogenes pathogen; and (b) reagents for performing a targeted sequencing reaction for detecting a Listeria monocytogenes pathogen. In some embodiments, the reagents for performing a PCR amplification reaction comprise at least one pair of Listeria monocytogenes specific primers. In some embodiments, the reagents for performing a PCR amplification reaction comprise multiple pairs of Listeria monocytogenes specific primers. In some embodiments, the at least one pair of Listeria monocytogenes specific primers. In some embodiments, the reagents for performing the targeted sequencing reaction are specific for detection of Listeria. In some embodiments, the reagents for the targeted sequencing reaction comprise reagents for a pore sequencing reaction. In some embodiments, the reagents for the targeted sequencing reaction comprises specifically designed primers. In some embodiments, the kit further comprises at least one of Library Reagent 3, Library Reagent 7, or any one of Library Reagents 8-20. In some embodiments, the kit further comprises written instructions for use of the kit on the food or the environmental samples.

In some aspects, the present disclosure provides for a method comprising: (a)

performing a PCR amplification reaction on a food or environmental sample from a food processing facility, wherein the PCR reaction amplifies at least one gene from a Listeria monocytogenes pathogen; and (b) performing a sequencing reaction on a food or environmental sample from a food processing facility, wherein the sequencing reaction detects a plurality of genes from a Listeria monocytogenes pathogen; (c) calculating the genetic distance between Listeria positive samples; and (d) mapping the genetic distance calculated in step c) the latter across space and time to one or more physical locations within the food processing facility. In some embodiments, the genetic distance is determined by calculating a number of unique nucleic acid base pairs between Listeria positive samples.

In some aspects, the present disclosure provides for a method comprising: (a) performing a PCR amplification reaction on a plurality of food or environmental samples from a plurality of physical locations within a facility, wherein said PCR reaction amplifies at least one gene from a Listeria spp. bacterium thereby generating a plurality of amplification products containing said at least one gene; (b) performing a sequencing reaction on said plurality of amplification products, wherein said sequencing reaction detects a plurality of genes from a Listeria spp. bacterium; (c) calculating at least a pairwise genetic distance between at least two genes among said plurality of genes detected from said Listeria spp. bacterium, wherein said at least two genes represent at least two of said plurality of physical locations within said facility; and (d) associating, via a computer, said at least a pairwise genetic distance calculated in (c) to said at least two of said plurality of physical locations within said facility.

In some aspects, the present disclosure provides for a method comprising: (a) performing a PCR amplification reaction on a plurality of food or environmental samples from a plurality of physical locations within a facility, wherein said PCR reaction amplifies at least one gene from a Listeria spp. bacterium to generate a plurality of spatially-addressable amplification products containing said at least one gene; (b) performing a sequencing reaction on said plurality of amplification products, wherein said sequencing reaction detects a gene characteristic to a particular Listeria spp. bacterium within said plurality of spatially-addressable amplification products; and (d) associating, via a computer, the presence of said particular Listeria spp. bacterium with at least one of said plurality of physical locations within said facility via said spatially-addressable amplification product.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1: is a Venn diagram illustrating a process that can simultaneously identify: a) a listeria species; b) whether it is a resident versus a transient species; and c) conduct environmental mapping of the species.

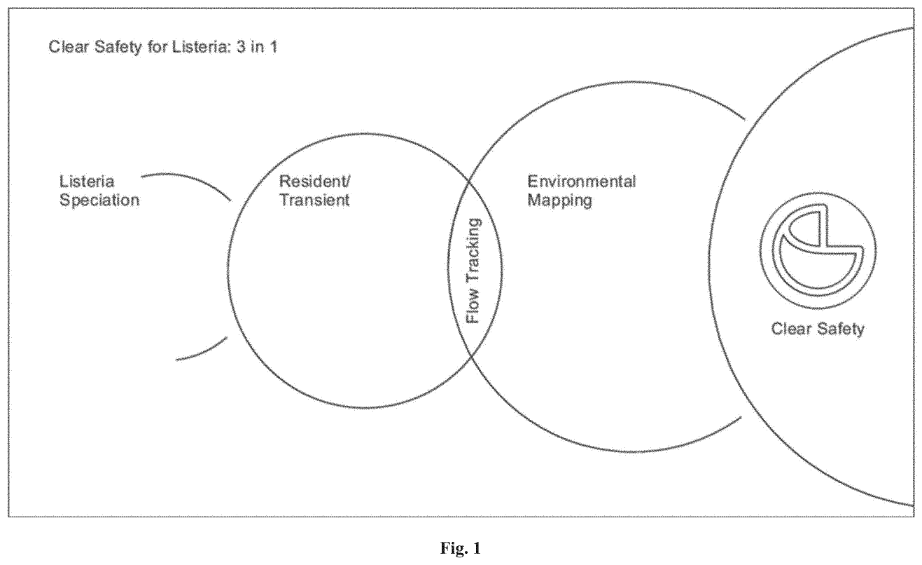

FIG. 2: illustrates the environmental monitoring step of a screen for Listeria. The top side of the figure illustrates the identification of Listeria in the environment.

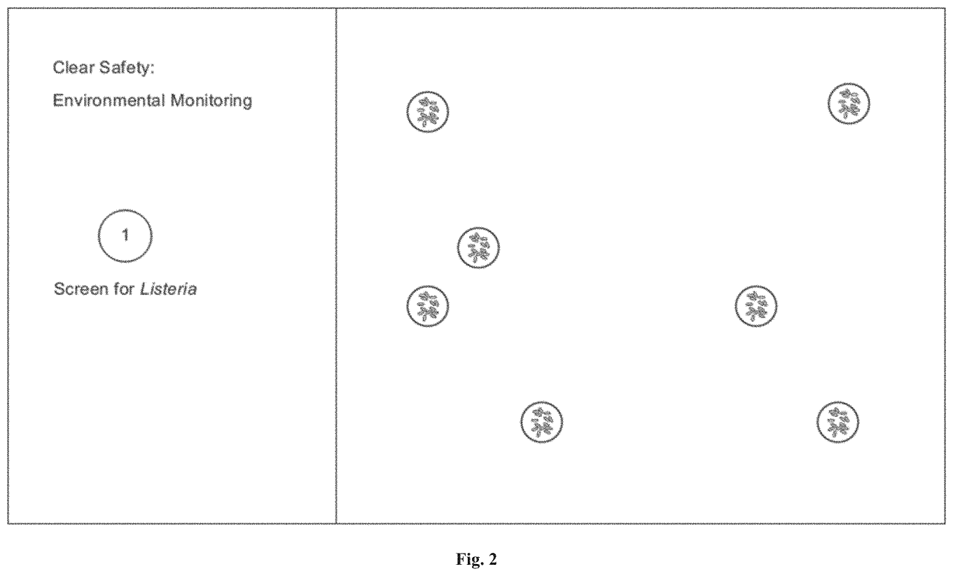

FIG. 3: illustrates the mapping step of a screen for Listeria. The top side of the figure illustrates an overlay of the Listeria identified in step 1 with environmental locations (i.e., mapping step).

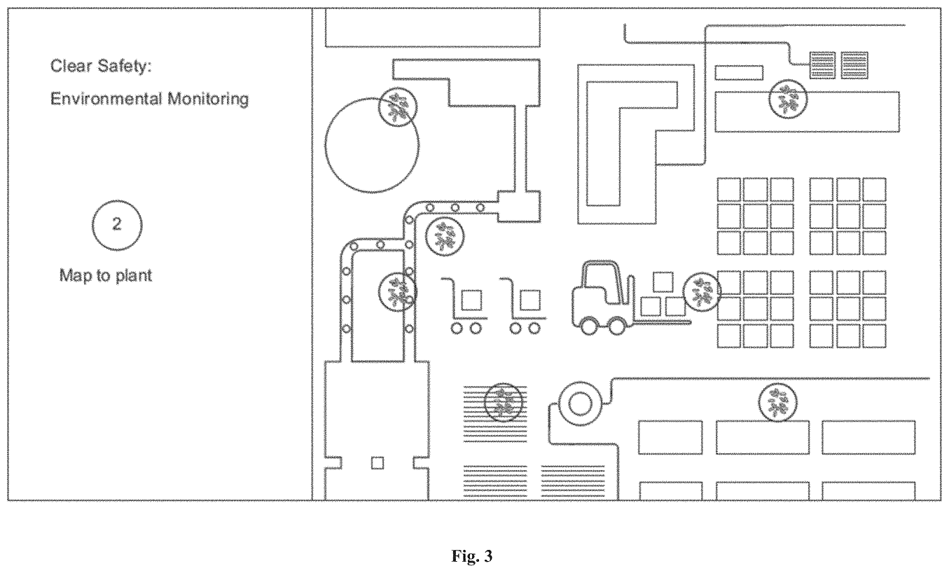

FIG. 4: illustrates the relatedness step of a screen for Listeria. Broken circles represent highly identical species. Solid circles represent highly identical species. Partially broken circles represent highly identical species. The overlay of each species with its environmental location provides an identification of each species and strain present at a given location.

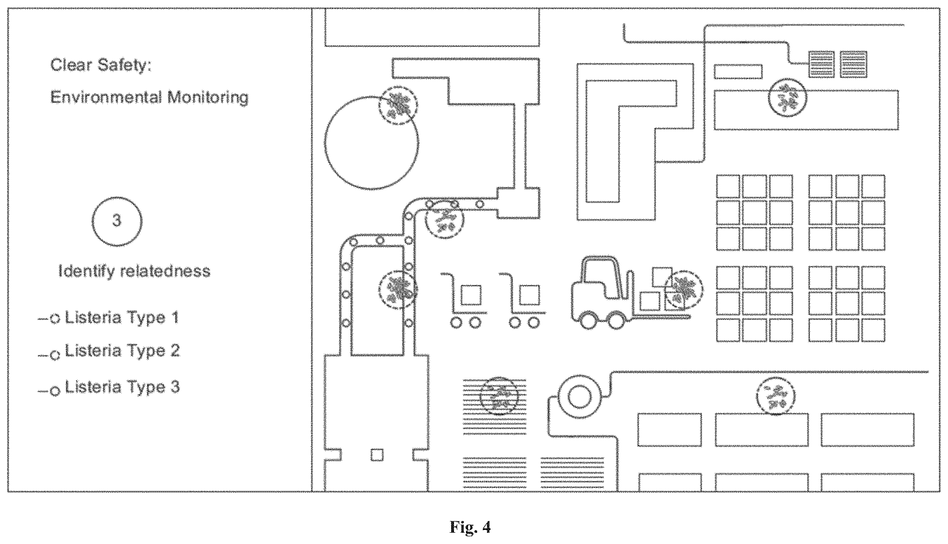

FIG. 5: illustrates the metadata step of a screen for Listeria. In this step, metadata is used to correlate the date and the time where each species or strain of listeria is identified at a certain location.

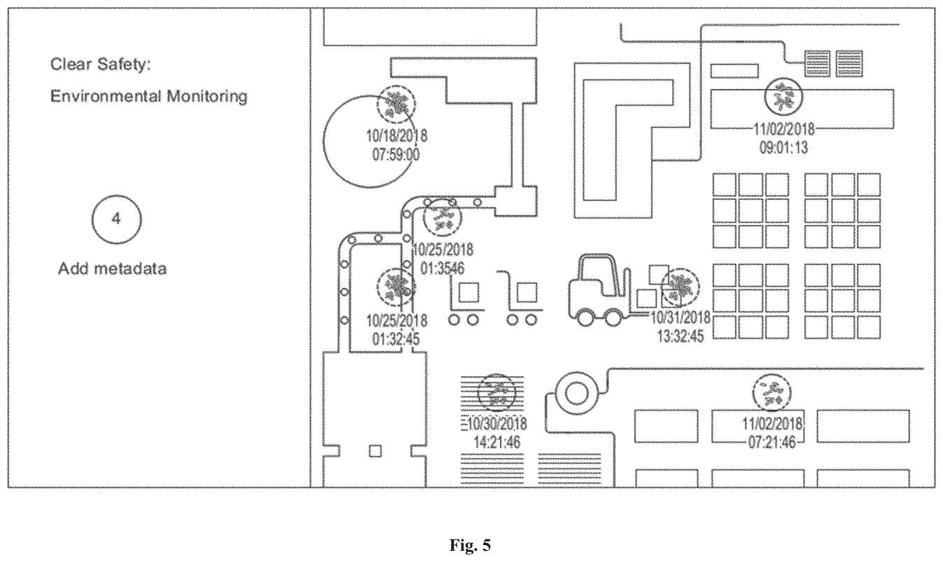

FIG. 6: illustrates how a process of the disclosure can be used to track the flow of a pathogen.

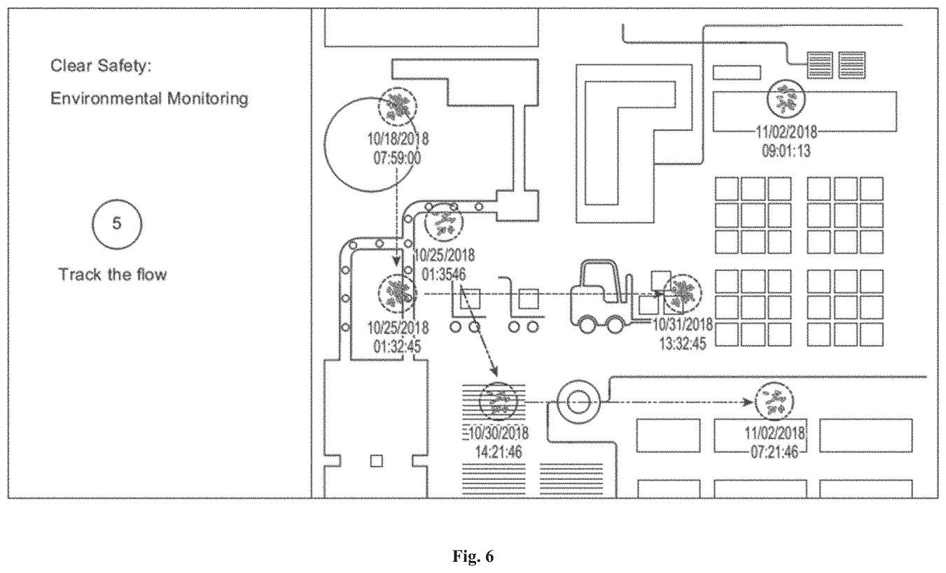

FIG. 7: illustrates a transmission of an electronic communication comprising a data set associated with a sequencing reaction from one or more food processing facilities to a server.

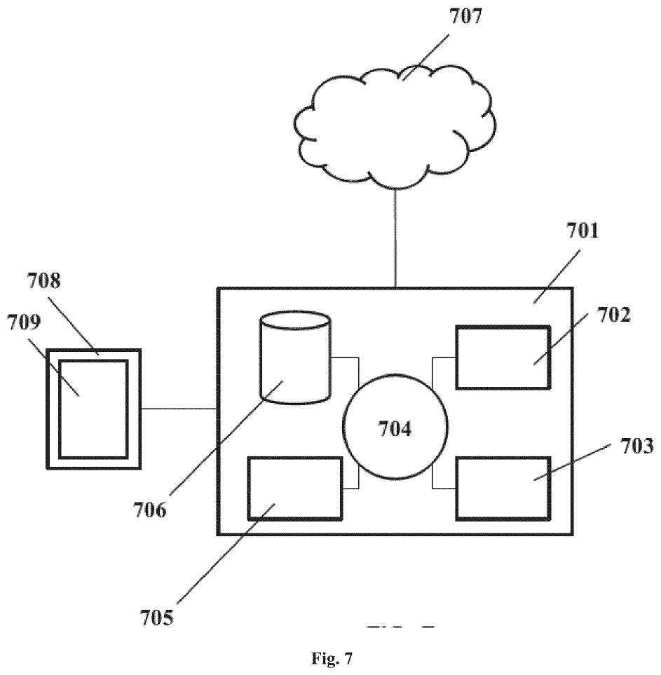

FIG. 8: is a picture showing a flow cell.

FIG. 9: is a picture showing a priming port of the flow cell.

FIG. 10: illustrates slowly aspirating an air bubble and a small amount of preservative buffer within the flow cell.

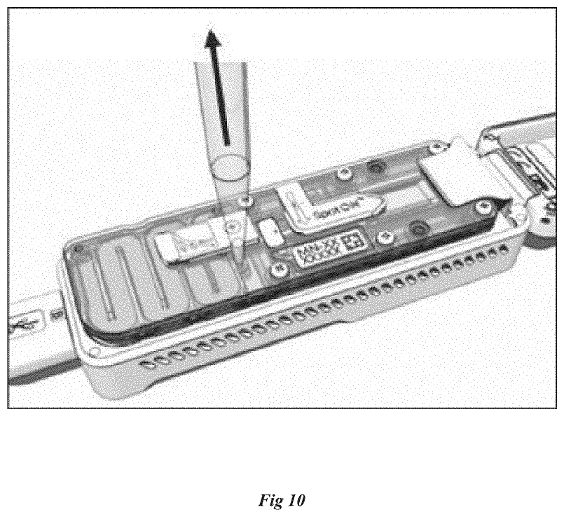

FIG. 11: is a picture illustrating slowly dispensing 800 μL of Priming Mix into the Priming Port of the flow cell, ensuring the pipette tip is seated well inside the Priming Port and remains vertical.

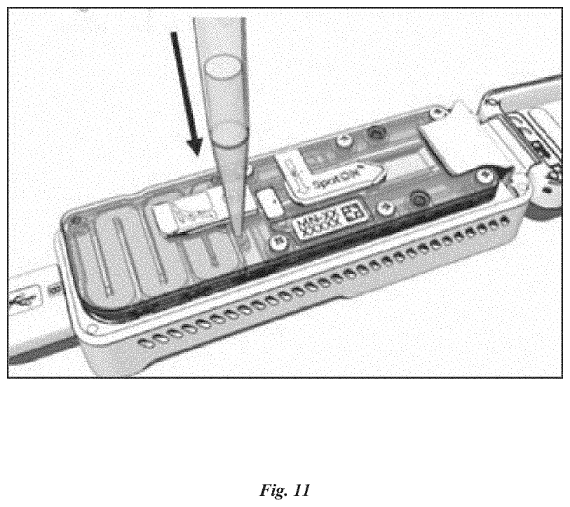

FIG. 12: is a picture illustrating how the Final Library Loading Mix is pipetted into the SpotON port of the flow cell, ensuring the solution is not directly pipetted into the port, but rather drops are formed and allowed to drop into the port

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Food processing facilities, companies and establishments typically employ an environmental sampling program to monitor for food spoilage microorganisms and food poisoning pathogens. Such program can enable the detection of unacceptable microbial contamination in a timely manner. Sampling programs should include the collection of samples during production on a regular basis from work surfaces in a randomized manner which reflect the differing working conditions. In addition, samples should be taken from these sites after sanitizing and from sites which may serve as harbors of resident organisms.

From a food processing facility's perspective, the presence of foodborne pathogens is important to product quality control as well as infrastructure maintenance. This information has traditionally been used to redirect or withhold product and ultimately, to sanitize equipment. As new tools become available, they have empowered managers to leverage test results for purposes that transcend product fate. For instance, the ability to estimate the genetic distance between samples across time and space, enables one to distinguish transient pathogens from those that have not been eradicated following a prior contamination event (resident pathogens). On the one hand, this information allows managers to infer the source of the adulterant while on the other hand, this information allows managers to identify compartments that demand comprehensive decontamination.

In food processing facilities sampling should not only be conducted on food contact surfaces, but the evaluation of non-food contact surfaces such as conveyor belts, rollers, walls, drains and air is equally as important as there are many ways (aerosols and human intervention) in which microorganisms can migrate from non-food contact surfaces to food. The results of these samples should be tabulated as soon as available and in such a way that they can be compared with previous results in order to highlight trends, so that adulterated foods or environmental locations can be identified.

Many different disease-causing microorganisms can contaminate foods, and there are many different foodborne infections. Although our scientific understanding of pathogenic microorganisms and their toxins is continually advancing, some of the most common microorganisms associated with foodborne illnesses include microorganisms of the Salmonella, Campylobacter, Listeria, and Escherichia genus.

Salmonella for example is widely dispersed in nature. It can colonize the intestinal tracts of vertebrates, including livestock, wildlife, domestic pets, and humans, and may also live in environments such as pond-water sediment. It is spread through the fecal-oral route and through contact with contaminated water. (Certain protozoa may act as a reservoir for the organism). It may, for example, contaminate poultry, red meats, farm-irrigation water (thereby contaminating produce in the field), soil and insects, factory equipment, hands, and kitchen surfaces and utensils.

Campylobacter jejuni is estimated to be the third leading bacterial cause of foodborne illness in the U.S. The symptoms this bacterium causes generally last from 2 to 10 days and, while the diarrhea (sometimes bloody), vomiting, and cramping are unpleasant, they usually go away by themselves in people who are otherwise healthy. Raw poultry, unpasteurized (“raw”) milk and cheeses made from it, and contaminated water (for example, unchlorinated water, such as in streams and ponds) are major sources, but C. jejuni also occurs in other kinds of meats and has been found in seafood and vegetables.

Although the number of people infected by foodborne Listeria is comparatively small, this bacterium is one of the leading causes of death from foodborne illness. It can cause two forms of disease. One can range from mild to intense symptoms of nausea, vomiting, aches, fever, and, sometimes, diarrhea, and usually goes away by itself. The other, more deadly, form occurs when the infection spreads through the bloodstream to the nervous system (including the brain), resulting in meningitis and other potentially fatal problems.

Escherichia microorganisms are also diverse in nature. For instance, at least four groups of pathogenic Escherichia coli have been identified: a) Enterotoxigenic Escherichia coli (ETEC), b) Enteropathogenic Escherichia coli (EPEC), c) Enterohemorrhagic Escherichia coli (EHEC), and Enteroinvasive Escherichia coli (EIEC). While ETEC is generally associated with traveler's diarrhea some members of the EHEC group, such as E. coli 0157:H7, can cause bloody diarrhea, blood-clotting problems, kidney failure, and death. Thus, it is important to be able not only to identify individual microorganism, but also to distinguish them.

Provided herein are methods and apparatus for the identification of transient versus resident pathogenic and non-pathogenic microorganisms in food and environmental samples. The disclosure solves challenges in environmental monitoring by providing one process track the flow of pathogens in a mapped location and identify them as resident versus transient.

As used herein, the term “food processing facility” includes facilities that manufacture, process, pack, or hold food in any location globally. A food processing facility can, for example, determine the location and source of an outbreak of food-borne illness or a potential bioterrorism incident.

As used herein, the term “food” includes any nutritious substance that people or animals eat or drink, or that plants absorb, in order to maintain life and growth. Non-limiting examples of foods include red meat, poultry, fruits, vegetables, fish, pork, seafood, dairy products, eggs, egg shells, raw agricultural commodities for use as food or components of food, canned foods, frozen foods, bakery goods, snack food, candy (including chewing gum), dietary supplements and dietary ingredients, infant formula, beverages (including alcoholic beverages and bottled water), animal feeds and pet food, and live food animals. The term “environmental sample,” as used herein, includes all food contact substances or items from a food processing facility. The term environmental sample includes a surface swab of a food contact substance, a surface rinse of a food contact substance, a food storage container, a food handling equipment, a piece of clothing from a subject in contact with a food processing facility, or another suitable sample from a food processing facility.

The term “sample” as used herein, generally refers to any sample that can be informative of an environment or a food, such as a sample that comprises soil, water, water quality, air, animal production, feed, manure, crop production, manufacturing plants, environmental samples or food samples directly. The term “sample” may also refer to other non-food sample, such as samples derived from a subject, such as comprise blood, plasma, urine, tissue, faces, bone marrow, saliva or cerebrospinal fluid. Such samples may be derived from a hospital or a clinic.

As used herein, the term “subject,” can refer to a human or to another animal. An animal can be a mouse, a rat, a guinea pig, a dog, a cat, a horse, a rabbit, and various other animals. A subject can be of any age, for example, a subject can be an infant, a toddler, a child, a pre-adolescent, an adolescent, an adult, or an elderly individual.

As used herein, the term “disease,” generally refers to conditions associated with the presence of a microorganism in a food, e.g., outbreaks or incidents of foodborne disease.

The term “nucleic acid” or “polynucleotide,” as used herein, refers to a polymeric form of nucleotides of any length, either ribonucleotides or deoxyribonucleotides. Polynucleotides include sequences of deoxyribonucleic acid (DNA), ribonucleic acid (RNA), or DNA copies of ribonucleic acid (cDNA).

The term “polyribonucleotide,” as used herein, generally refers to polynucleotide polymers that comprise ribonucleic acids. The term also refers to polynucleotide polymers that comprise chemically modified ribonucleotides. A polyribonucleotide can be formed of D-ribose sugars, which can be found in nature, and L-ribose sugars, which are not found in nature.

The term “polypeptides,” as used herein, generally refers to polymer chains comprised of amino acid residue monomers which are joined together through amide bonds (peptide bonds). The amino acids may be the L-optical isomer or the D-optical isomer.

The term “barcode,” as used herein, generally refers to a label, or identifier, that conveys or is capable of conveying information about one or more nucleic acid sequences from a food sample or from an environmental sample associated with said food sample. A barcode can be part of a nucleic acid sequence. A barcode can be independent of a nucleic acid sequence. A barcode can be a tag attached to a nucleic acid molecule. A barcode can have a variety of different formats. For example, barcodes can include: polynucleotide barcodes; random nucleic acid and/or amino acid sequences; and synthetic nucleic acid and/or amino acid sequences. A barcode can be added to, for example, a fragment of a deoxyribonucleic acid (DNA) or ribonucleic acid (RNA) sample before, during, and/or after sequencing of the sample. Barcodes can allow for identification and/or quantification of individual sequencing-reads. Examples of such barcodes and uses thereof, as may be used with methods, apparatus and systems of the present disclosure, are provided in U.S. Patent Pub. No. 2016/0239732, which is entirely incorporated herein by reference. In some instances, as described herein, a “molecular index” can either be a barcode itself or it can be a building block, i.e., a component or portion of a larger barcode.

The term “sequencing,” as used herein, generally refers to methods and technologies for determining the sequence of nucleotide bases in one or more nucleic acid polymers, i.e., polynucleotides. Sequencing can be performed by various systems currently available, such as, without limitation, a sequencing system by Illumina®, Pacific Biosciences (PacBio®), Oxford Nanopore®, Genia (Roche) or Life Technologies (Ion Torrent®). Alternatively, or in addition, sequencing may be performed using nucleic acid amplification, polymerase chain reaction (PCR) (e.g., digital PCR, quantitative PCR, or real time PCR), or isothermal amplification. Such systems may provide a plurality of raw data corresponding to the genetic information associated with a food sample or an environmental sample. In some examples, such systems provide nucleic acid sequences (also “reads” or “sequencing reads” herein). The term also refers to epigenetics which is the study of heritable changes in gene function that do not involve changes in the DNA sequence. A read may include a string of nucleic acid bases corresponding to a sequence of a nucleic acid molecule that has been sequenced.

As used herein, the term “spatially-addressable” when used to refer to a nucleic acid refers to a nucleic acid associated with a specific location in space. Spatially-addressable nucleic acids can be mapped to a location of origin which can be tracked throughout subsequent manipulations. In some embodiments, spatially-addressable nucleic acids are spatially addressable by virtue of a barcode or a unique nucleotide sequence appended thereto which is associated with a location. In some embodiments, spatially-addressable nucleic acids are spatially addressable via the addition of a unique chemical moiety (e.g. a fluor, a dye, a mass tag, a chemically unique nucleic acid derivative such as an LNA or a morpholino) appended thereto. The appending can occur via a variety of methods, including e.g. enzymatic ligation, chemical coupling, and polymerase chain reaction. In some embodiments, “spatially-addressable” nucleic acids are directly spatially addressable, there being a direct association (e.g. via a database in a computer system) between said nucleic acid and said location. In some embodiments, “spatially-addressable” nucleic acids are indirectly spatially-addressable, there being an association between said nucleic acid and a particular sample id, and an association between a particular sample id and said location.

As used herein, the term “pathogen” refers to any agent that causes or promotes diseases or illnesses in animals, and particularly in humans, such pathogens including those of parasitic, viral bacterial, or archaeal origin. In some embodiments, a microorganism that can injure its host, e.g., by competing with it for metabolic resources, destroying its cells or tissues, or secreting toxins can be considered a pathogenic microorganism. In some embodiments, the pathogen is a foodborne or zoonotic pathogen. Description of major foodborne pathogens can be found e.g. in World Health Organization (WHO) Foodborne Disease Burden Epidemiology Reference Group 2007-2015. World Health Organization; Geneva, Switzerland: 2015. WHO estimates of the global burden of foodborne diseases (ISBN 978 92 4 156516 5). Foodborne or zoonotic pathogens include, but are not limited to, Norovirus, Hepatitis A virus, Campylobacter spp. (including e.g. C. jejuni subs. jejuni and C. coli), pathogenic E. coli (including e.g. Enteropathogenic E. coli—EPEC, Enteropathogenic E. coli—ETEC, and Shiga toxin-producing E. coli—STEC), Yersinia spp. (including e.g. Y. enterocolitica), Salmonella spp. (including S. enterica and non-typhoidal S. enterica, Salmonella Paratyphi A, Salmonella Paratyphi B, and Salmonella Paratyphi C, and Salmonella Typhi), Shigella spp., Vibrio spp. (including V. cholerae), Brucella spp., Listeria spp. (including Listeria monocytogenes and other Listeria species or strains described herein), Mycobacterium spp. (including e.g. Mycobacterium bovis), Cryptosporidium spp., Entamoeba spp. (including e.g. E. histolytica), Giardia spp., Toxoplasma spp. (including e.g. Toxoplasma gondii), helminths, Echinococcus spp. (including e.g. E. granulosus and E. multilocularis), Taenia spp. (includin e.g. Taenia solium), Ascaris spp., Trichinella spp., Clonorchis spp. (including e.g. Clonorchis sinensis), Fasciola spp, intestinal flukes, Opisthorchis spp., Paragonimus spp, Bacillus anthracis, Balantidium coli, Francisella Tularensis, Sarcocystis spp. (including e.g. S. hominis, S. suihominis, and S. nesbitti), Taenia spp. (including e.g. T. solium and T. saginata), Trichinella spp. (including e.g. T. spiralis, T nativa, T. britovi and T. pseudospiralis).

In some embodiments, the pathogen is an opportunistic pathogen (e.g. a pathogen contributing to nosocomial infections, a hospital-resident pathogen, or a clinical-location-resident pathogen). Such pathogens are described, e.g. in Dasgupta et al. Indian J Crit Care Med. 2015 January; 19(1): 14-20. Such pathogens include, but are not limited to, Pseudomonas spp. (including e.g. Pseudomonas aeruginosa and multidrug-resistant variants thereof), Escherichia coli (including e.g. uropathogenic variants thereof such as sequence type 131), Candida spp. (including e.g. C. albicans, C. tropicalis, C. glabrata, C. parapsilosis, C. kefyr, C. dubliniensis, and C. parasilosis), Klebsiella spp. (including e.g. K. pneumoniae and subspecies thereof such as pneumoniae, ozaenae, and rhinoscleromatis; K oxytoca; K terrigena; K planticola, and K. ornithinolytica), Enterococcus spp. (including e.g. E. faecalis and E. faecium), Acinetobacter spp. (including e.g. A. baumannii), Burkholderia spp. (including e.g. B. cepacia), coagulase-negative staphylococci, Enterobacter spp. (including e.g. E. cloacae and E. aerogenes), Stenotrophomonas spp. (including e.g. S. maltophilia), F.

As used herein, the term “genetic distance” shall be understood as a measure of the genetic divergence between two genes (e.g. to paralogous or orthologous genes from two different species or strains), two species, two genomes or two populations. The genetic distance, e.g., between different species, can be determined by suitable methods including but not limited to determining the Nei's standard distance (see e.g. Nei, M. (1972). “Genetic distance between populations”. Am. Nat. 106: 283-292, which is incorporated by reference herein), the Goldstein distance (see e.g. L. L. Cavalli-Sforza; A. W. F. Edwards (1967). “Phylogenetic Analysis—Models and Estimation Procedures”. The American Journal of Human Genetics. 19 (3 Part I (May)) which is incorporated by reference herein), Reynolds/Weir/Cockerham's genetic distance (see e.g., John Reynolds; B. S. Weir; C. Clark Cockerham (November 1983) “Estimation of the coancestry coefficient: Basis for a short-term genetic distance”. Genetics. 105: 767-779, which is incorporated by reference herein), Nei's DA distance (see e.g. Nei, M., F. Tajima, & Y. Tateno (1983) Accuracy of estimated phylogenetic trees from molecular data. II. Gene frequency data. J. Mol. Evol. 19:153-170, which is incorporated by reference herein), the Euclidian distance (see e.g. Nei, M. (1987). Molecular Evolutionary Genetics. (Chapter 9). New York: Columbia University Press., which is incorporated by reference herein), the 1995 variant of the Goldstein distance (see e.g. Gillian Cooper; William Amos; Richard Bellamy; Mahveen Ruby Siddiqui; Angela Frodsham; Adrian V. S. Hill; David C. Rubinsztein (1999). “An Empirical Exploration of the (δμ²) Genetic Distance for 213 Human Microsatellite Markers”. The American Journal of Human Genetics. 65: 1125-1133, which is incorporated by reference herein), the 1973 variant of Nei's minimum genetic distance (see e.g. Nei, M.; A. K. Roychoudhury (1974). “Genic variation within and between the three major races of man, Caucasoids, Negroids, and Mongoloids”. The American Journal of Human Genetics. 26: 421-443, which is incorporated by reference herein), or the 1972 variant of Roger's distance (see e.g. Rogers, J. S. (1972). Measures of similarity and genetic distance. In Studies in Genetics VII. pp. 145-153. University of Texas Publication 7213. Austin, Tex., which is incorporated by reference herein). Genetic distance can be calculated using suitable software including but not limited to GENDIST (see e.g. Felsenstein, J. (1981). “Evolutionary trees from DNA sequences: A maximum likelihood approach”. Journal of Molecular Evolution. 17 (6): 368-376, which describes the PHYLIP package that implements GENDIST and is incorporated by reference herein), TFPGA, GDA, POPGENE, POPTREE2, and DISPAN. The “genetic similarity” is high when the genetic distance is low.

### Identifying Transient Versus Resident Pathogens

Disclosed herein are methods and apparatuses that allow the distinction of a microorganism that has been newly introduced into a food processing facility or any other environmental setting in which tracking hygiene is critical, such as a hospital or a clinic. In some instances, resident microorganisms reflect a persistent contamination within a location, e.g., a food processing facility or a hospital, that is very different than the transient pathogens that are being repeatedly introduced into the locations. Discriminating resident and transient pathogens provides more clarity for differentiation of source of contaminations and intervention strategies. This strategy can be used, for example, to manage contaminations with managing contaminations with Listeria monocytogenes. For example, Campylobacter is part of the natural gut microflora of most food-producing animals, such as chickens, turkeys, swine, cattle, and sheep. Typically, each contaminated poultry carcass can carry from about 100 to about 100,000 Campylobacter cells. On one hand, given the fact that less than 500 Campylobacter cells can cause infection, poultry products pose a significant risk for consumers who mishandle fresh or processed poultry during preparation or who undercook it. On another hand, one must be able to distinguish a normal level of e.g. a Campylobacter on a food carcass from a Campylobacter overgrowth in a sample or from the presence of a new strain of Campylobacter in a food processing facility, environment, or food sample. One must also be able to identify a new source of contamination in a facility from existing sources.

In some embodiments, identification of a transient pathogen involves the detection of a new species or a new strain of a pathogen not previously detected in a facility. In some embodiments, identification of a transient pathogen involves determination of genetic distances between at least one gene in a pathogen at different times to determine a background rate of mutation of a resident pathogen, and then distinguishing a transient pathogen via a genetic distance representing a rate of mutation higher than the determined background rate of mutation. In some embodiments, identification of a transient pathogen involves determination of genetic distances among at least three genes from a pathogen at least two different sampling times, clustering said genes according to said genetic distances, and identifying introduction of a transient pathogen via presence of a new cluster of genes that occurs at a third sampling time.

In some instances, the methods disclosed herein further comprise performing an additional assay to confirm the presence of the pathogenic microorganism in the sample, such as a serotyping assay, a polymerase chain reaction (PCR) assay, an enzyme-linked immunosorbent (ELISA) assay, or an enzyme-linked fluorescent assay (ELFA) assay, restriction fragment length polymorphisms (RFLP) assay, pulse field gel electrophoresis (PFGE) assay, multi-locus sequence typing (MLST) assay, targeted DNA sequencing assay, whole genome sequencing (WGS) assay, or shotgun sequencing assay.

In some aspects, the disclosure provides a method comprising obtaining a first plurality of nucleic acid sequences from a first sample of a food processing facility; creating a data file in a computer that associates one or more of said first plurality of nucleic acid sequences with said food processing facility; obtaining a second plurality of nucleic acid sequences from a second food sample of said food processing facility; and scanning a plurality of sequences from said second plurality of nucleic acid sequences for one or more sequences associated with said food processing facility in the created data file.

One or more data files can be created that associate a microorganism with a food processing facility. In some instances, a data file can provide a collection of sequencing reads that can be associated with one or more strains of a microorganism present in the processing facility. In some cases, more than 10, 15, 20, 25, 30, 35, 40, 45, 50, 60, 70, 80, 90, 100, or 1000 bacterial strains can be associated with one or more food processing facilities.

A computer system 701 can be programmed or otherwise configured to process and transmit a data set from a food processing facility, food testing labs, or any other diagnostic labs. The computer system 701 includes a central processing unit (CPU, also “processor” and “computer processor” herein) 704, which can be a single core or multi core processor, or a plurality of processors for parallel processing. The computer system 701 also includes memory or memory location 705 (e.g., random-access memory, read-only memory, flash memory), electronic storage unit 706 (e.g., hard disk), communication interface 702 (e.g., network adapter) for communicating with one or more other systems, such as for instance transmitting a data set associated with said sequencing reads, and peripheral devices 704, such as cache, other memory, data storage and/or electronic display adapters. The memory 705, storage unit 706, interface 702 and peripheral devices 703 are in communication with the CPU 704 through a communication bus (solid lines), such as a motherboard. The storage unit 706 can be a data storage unit (or data repository) for storing data. For instance, in some cases, the data storage unit 706 can store a plurality of sequencing reads and provide a library of sequences associated with one or more strains from one or more microorganisms associated with a food processing facility, food testing labs, or any other diagnostic labs.

The computer system 701 can be operatively coupled to a computer network (“network”) 707 with the aid of the communication interface 702. The network 707 can be the Internet, an internet and/or extranet, or an intranet and/or extranet that is in communication with the Internet. The network 707 in some cases is a telecommunication and/or data network. The network 707 can include one or more computer servers, which can enable distributed computing, such as cloud computing. The network 707, in some cases with the aid of the computer system 701, can implement a peer-to-peer network, which may enable devices coupled to the computer system 701 to behave as a client or a server.

Identification of a Contamination Source within a Facility, or Mapping a Contamination to a Location in a Facility

Disclosed herein are methods and apparatuses that allow for the tracing/identification of a contamination source or contamination spread of a microbial organism within any of the facilities described herein. In some instances, such a method involves first performing sequencing reactions on nucleic acids of microbes obtained from samples from multiple locations in a facility, determination of genetic distances between paralogous/orthologous microbe genes within the samples, ranking the paralogous/orthologous microbe genes within the samples according to the genetic distance, and identifying a first source of contamination from the ranking. In some cases, the paralogous/orthologous microbe genes within the samples are first clustered, and then ranked within the clusters to determine more than one first source of contamination.

In some cases, the microbe gene is a ribosomal or ribosomal associated gene. Such genes include, but are not limited to, 16S rRNA genes, rps genes, and rpl genes. In some embodiments, such genes are selected from a ribosomal protein L1p, L2p, L3p, L4p, L5p, L6p, L10p, L11p, L12p, L13p, L14p, L15p, L18p, L22p, L23p, L24p, L29p, L30p, S2p, S3p, S4p, S5p, S7p, S8p, S9p, S10p, S11p, S12p, S13p, S14p, S15p, S17p, S19p, and L7ae gene; a ribosomal protein L9p, L16p, L17p, L19p, L20p, L21p, L25p, L27p, L28p, L31p, L32p, L33p, L34p, L35p, L36p, S1p, S6p, S16p, S18p, S20p, S21p, S22p, and S31e gene; a ribosomal protein L10e, L13e, L14e, L15e, LXa/L18ae, L18e, L19e, L21e, L24e, L30e, L31e, L32e, L34e, L35ae, L37ae, L37e, L38e, L39e, L40e, L41e, L44e, S17e, S19e, S24e, S25e, S26e, S27ae, S27e, S28e, S30e, S3ae, S4e, S6e, S8e, L45a, L46a, and L47a gene. In some embodiments, such genes are selected from a ribosomal protein L1p, L2p, L3p, L4p, L5p, L6p, L10p, L11p, L12p, L13p, L14p, L15p, L18p, L22p, L23p, L24p, L29p, L30p, S2p, S3p, S4p, S5p, S7p, S8p, S9p, S10p, S11p, 512p, 513p, 514p, 515p, S17p, 519p, and L7ae gene. In some embodiments, such genes are selected from a ribosomal protein L9p, L16p, L17p, L19p, L20p, L21p, L25p, L27p, L28p, L31p, L32p, L33p, L34p, L35p, L36p, S1p, S6p, S16p, S18p, S20p, S21p, S22p, and S31e gene. In some embodiments, such genes are selected from a ribosomal protein L10e, L13e, L14e, L15e, LXa/L18ae, L18e, L19e, L21e, L24e, L30e, L31e, L32e, L34e, L35ae, L37ae, L37e, L38e, L39e, L40e, L41e, L44e, S17e, S19e, S24e, S25e, S26e, S27ae, S27e, S28e, S30e, S3ae, S4e, S6e, S8e, L45a, L46a, and L47a gene.

In some aspects, the present disclosure provides for a method comprising: (a) performing a PCR amplification reaction on a plurality of food or environmental samples from a plurality of physical locations within a facility, wherein the PCR reaction amplifies at least one gene from a Listeria spp. bacterium thereby generating a plurality of amplification products containing the at least one gene; (b) performing a sequencing reaction on the plurality of amplification products, wherein the sequencing reaction detects a plurality of genes from a Listeria spp. bacterium; (c) calculating at least a pairwise genetic distance between at least two genes among the plurality of genes detected from the Listeria spp. bacterium, wherein the at least two genes represent at least two of the plurality of physical locations within the facility; and (d) associating, via a computer, the at least a pairwise genetic distance calculated in (c) to the at least two of the plurality of physical locations within the facility. In some cases, the at least a pairwise genetic distance in (c) is determined at least in part by calculating a number of unique nucleic acid base pairs between the at least two genes among the plurality of genes detected from the Listeria spp. bacterium. In some cases, the at least a pairwise genetic distance in (c) is a Nei's standard distance, a Goldstein distance, a Reynolds/Weir/Cockerham's genetic distance, a Roger's distance, or a variant thereof. In some cases, the at least two genes are orthologous genes of at least two Listeria strains or species. In some cases, (a) generates a plurality of amplification products that are respectively spatially-addressable to the one or more physical locations within the facility. In some cases, (a) comprises performing the PCR amplification the plurality of samples utilizing oligonucleotide amplification primers containing unique sequences that are spatially addressable to the physical locations within the facility. In some cases, the method comprises clustering the plurality of physical locations into at least one cluster having a common contamination origin of Listeria spp. contamination according to the at least pairwise genetic distance. In some cases, the method comprises ranking the one or more physical locations within the facility according to the genetic distance associated in (d) to determine a trajectory of Listeria spp. contamination between two or more locations within the facility or a common contamination origin of Listeria spp. contamination among the two or more locations within the facility. In some cases, the facility is a food processing facility, a hospital, a pharmacy, a medical facility, or a clinical facility.

Also disclosed herein are methods and apparatuses that allow for the mapping of microbial organism contamination to a location within any of the facilities described herein. In some instances, the method comprises: (a) performing a PCR amplification reaction on a plurality of food or environmental samples from a plurality of physical locations within a facility, wherein the PCR reaction amplifies at least one gene from a Listeria spp. bacterium to generate a plurality of spatially-addressable amplification products containing the at least one gene; (b) performing a sequencing reaction on the plurality of amplification products, wherein the sequencing reaction detects a gene characteristic to a particular Listeria spp. bacterium within the plurality of spatially-addressable amplification products; and (c) associating, via a computer, the presence of the particular Listeria spp. bacterium with at least one of the plurality of physical locations within the facility via the spatially-addressable amplification product. In some cases, the method further comprises (d) outputting, via the computer, the at least one location contaminated with the particular Listeria spp. bacterium. In some cases, the particular Listeria spp. bacterium is a pathogenic Listeria strain or species.

### Pathogenic Microorganisms

As used herein, the term “pathogen” refers to any agent that causes or promotes diseases or illnesses in animals, and particularly in humans, such pathogens including those of parasitic, viral bacterial, or archaeal origin. In some embodiments, a microorganism that can injure its host, e.g., by competing with it for metabolic resources, destroying its cells or tissues, or secreting toxins can be considered a pathogenic microorganism. Examples of classes of pathogenic microorganisms include viruses, bacteria, mycobacteria, fungi, protozoa, and some helminths. In some aspects, the disclosure provides methods for detecting one or more microorganisms from a food sample or from an environment associated with said food sample—such as from a table, a floor, a boot cover, an equipment of a food processing facility—or from a food related sample that comprise soil, water, water quality, air, animal production, feed, manure, crop production, manufacturing plants, environmental samples, or non-food derived samples, such as samples from clinical sources that comprise blood, plasma, urine, tissue, faces, bone marrow, saliva or cerebrospinal fluid by analyzing a plurality of nucleic acid sequencing reads from such samples. In some embodiments, viruses include a DNA virus or a RNA virus. The virus may be, for example, a double stranded DNA virus, a single stranded DNA virus, a double stranded RNA virus, a positive sense single stranded RNA virus, a negative sense single stranded RNA virus, a single stranded RNA-reverse transcribing virus (retrovirus) or a double stranded DNA reverse transcribing virus. Examples of DNA viruses cam include, but are not limited to, cytomegalovirus, Herpes Simplex, Epstein-Barr virus, Simian virus 40, Bovine papillomavirus, Adeno-associated virus, Adenovirus, Vaccinia virus, and Baculovirus. Examples of RNA viruses can include, but are not limited to, Coronavirus, Semliki Forest virus, Sindbis virus, Poko virus, Rabies virus, Influenza virus, SV5, Respiratory Syncytial virus. Venezuela equine encephalitis virus, Kunjin virus, Sendai virus, Vesicular stomatitisvirus, and Retroviruses. Examples of coronaviruses include alphacoronavirus, betacoronavirus, deltacoronavirus, and gammacoronavirus. Further examples of coronavirus can include MERS-CoV, SARS-CoV, and SARS-Cov-2 (e.g., SARS-COV-2)

Many pathogenic microorganisms are further subdivided into serotypes, which can differentiate strains by their surface and antigenic properties. For instance, Salmonella species are commonly referred to by their serotype names. For example, Salmonella enterica subspecies enterica is further divided into numerous serotypes, including S. enteritidis and S. typhimurium. In some aspects, the methods of the disclosure can distinguish between such subspecies of a variety of Salmonella by analyzing their nucleic acid sequences.

Escherichia coli (E. coli) bacteria normally live in the intestines of people and animals. Many E. coli are harmless and in some aspects are an important part of a healthy human intestinal tract. However, many E. coli can cause illnesses, including diarrhea or illness outside of the intestinal tract and should be distinguished from less pathogenic strains. In some aspects, the methods of the disclosure can distinguish between various subspecies of a variety of Escherichia bacteria by analyzing their nucleic acid sequences.

Listeria is a genus containing harmful bacterial species that can be found in refrigerated, ready-to-eat foods (meat, poultry, seafood, and dairy—unpasteurized milk and milk products or foods made with unpasteurized milk) and produce harvested from soil contaminated with animal faeces. Pathogenic Listeria species known to be transmitted via this route include, for example, L. monocytogenes and L. ivanovii. Many animals can carry even pathogenic bacteria of this genus without appearing ill, which increases the challenges in identifying the pathogen derived from a food source. In addition, some species of Listeria can grow at refrigerator temperatures where most other foodborne bacteria do not, another factor that increases the challenges of identifying Listeria. When eaten, Listeria may cause listeriosis, an illness to which pregnant women and their unborn children are very susceptible. In some aspects, the methods of the disclosure can distinguish between various species Listeria genus bacteria (e.g. Listeria monocytogenes, Listeria seeligeri, Listeria ivanovii, Listeria welshimeri, Listeria marthii, Listeria innocua, Listeria grayi, Listeria fleischmannii, Listeria floridensis, Listeria aquatica, Listeria newyorkensis, Listeria cornellensis, Listeria rocourtiae, Listeria weihenstephanensis, Listeria grandensis, Listeria riparia, or Listeria booriae) by analyzing their nucleic acid sequences. In some cases, the species distinguished are pathogenic. Pathogenic species include, e.g. L. monocytogenes and L. ivanoviicases, the species distinguished are nonpathogenic. Nonpathogenic species include e.g. Listeria seeligeri, Listeria welshimeri, Listeria marthii, Listeria innocua, Listeria grayi, Listeria fleischmannii, Listeria floridensis, Listeria aquatica, Listeria newyorkensis, Listeria cornellensis, Listeria rocourtiae, Listeria weihenstephanensis, Listeria grandensis, Listeria riparia, and Listeria booriae.

Campylobacter jejuni is estimated to be the third leading bacterial cause of foodborne illness in the United States. Raw poultry, unpasteurized (“raw”) milk and cheeses made from it, and contaminated water (for example, unchlorinated water, such as in streams and ponds) are major sources of Campylobacter, but it also occurs in other kinds of meats and has been found in seafood and vegetables. In some aspects, the methods of the disclosure can distinguish between various subspecies of a variety of Campylobacter bacteria by analyzing their nucleic acid sequences.

Non-limiting examples of pathogenic microorganisms that can be detected with the methods of the disclosure include: pathogenic Escherichia coli group, including Enterotoxigenic Escherichia coli (ETEC), Enteropathogenic Escherichia coli (EPEC), Enterohemorrhagic Escherichia coli (EHEC), Enteroinvasive Escherichia coli (EIEC), Salmonella spp., Campylobacter jejuni, Listeria spp., pathogenic Listeria spp., nonpathogenic Listeria spp., L. monocytogenes, L. ivanovii, L. seeligeri, L. welshimeri, L. marthii, L. innocua, L. grayi, L. fleischmannii, L. floridensis, L. aquatica, L. newyorkensis, L. cornellensis, L. rocourtiae, L. weihenstephanensis, L. grandensis, L. riparia, and L. booriae, Yersinia enterocolitica, Shigella spp., Vibrio parahaemolyticus, Coxiella burnetii, Mycobacterium bovis, Brucella spp., Vibrio cholera, Vibrio vulnificus, Cronobacter, Aeromonas hydrophila and other spp., Plesiomonas shigelloides, Clostridium perfringens, Clostridium botulinum, Staphylococcus aureus, Bacillus cereus and other Bacillus spp., Streptococcus spp., Enterococcus, and others.

### Barcodes

Unique identifiers, such as barcodes, can be added to one or more nucleic acids isolated from a sample from a food processing facility, from a hospital or clinic, or from another source. In some embodiments, such identifiers provide spatial-, location-, sample-, or acquisition time-addressability to the nucleic acids isolated from a sample from a food processing facility, from a hospital or clinic, or from another source. Barcodes can be used to associate a sample with a source; e.g., to associate an environmental sample with a specific food processing facility or with a particular location within said food processing facility. Barcodes can also be used to identify a processing of a sample, as described in U.S. Patent Publication No. 2016/0239732 or International App. No. PCT/US2018/067750, each of which is incorporated herein by reference in its entirety.

One or more barcodes or block of barcodes may be added to a nucleic acid sequence from a food sample or another sample from a food processing facility, such as a first, a second, a third, or any subsequent sample. In some cases, 1, 2, 3, 4, 5, 6, 7, 8, 9, 10, 11, 12, 13, 14, 15, 16, 17, 18, 19, or 20 identical barcodes are added to such samples. In other cases, distinct barcodes are added to such samples. In some cases, 1, 2, 3, 4, 5, 6, 7, 8, 9, 10, 11, 12, 13, 14, 15, 16, 17, 18, 19, or 20 distinct barcodes are added to such samples. The serial addition of two or more barcodes, either identical in sequence or distinct in sequence, can provide an indexing of a sample that is used in its analyses. The presence of additional barcode or barcode blocks make the system more robust against any barcode manufacturing error and can also significantly reduce the chance of cross contamination between barcodes. In some cases, a barcode is added to a nucleic acid sequence comprising complementary DNA (cDNA) sequences, ribonucleic acid (RNA) sequences, genomic deoxyribonucleic acid (gDNA) sequences, or a mixture of cDNA, RNA, and gDNA sequences.

Barcodes can have a variety of lengths. In some instances a barcode is from about 3 to about 25 nucleotides in length, from about 3 to about 24 nucleotides in length, from about 3 to about 23 nucleotides in length, from about 3 to about 22 nucleotides in length, from about 3 to about 21 nucleotides in length, from about 3 to about 20 nucleotides in length, from about 3 to about 19 nucleotides in length, from about 3 to about 18 nucleotides in length, from about 3 to about 17 nucleotides in length, from about 3 to about 16 nucleotides in length, from about 3 to about 15 nucleotides in length, from about 3 to about 14 nucleotides in length, from about 3 to about 13 nucleotides in length, from about 3 to about 12 nucleotides in length, from about 3 to about 11 nucleotides in length, from about 3 to about 10 nucleotides in length, from about 3 to about 9 nucleotides in length, from about 3 to about 8 nucleotides in length, or from about 3 to about 7 nucleotides in length.

In some instances, a barcode is from about 4 to about 25 nucleotides in length, from about 4 to about 24 nucleotides in length, from about 4 to about 23 nucleotides in length, from about 4 to about 22 nucleotides in length, from about 4 to about 21 nucleotides in length, from about 4 to about 20 nucleotides in length, from about 4 to about 19 nucleotides in length, from about 4 to about 18 nucleotides in length, from about 4 to about 17 nucleotides in length, from about 4 to about 16 nucleotides in length, from about 4 to about 15 nucleotides in length, from about 4 to about 14 nucleotides in length, from about 4 to about 13 nucleotides in length, from about 4 to about 12 nucleotides in length, from about 4 to about 11 nucleotides in length, from about 4 to about 10 nucleotides in length, from about 4 to about 9 nucleotides in length, from about 4 to about 8 nucleotides in length, or from about 4 to about 7 nucleotides in length.

In some instances, a barcode is from about 5 to about 25 nucleotides in length, from about 5 to about 24 nucleotides in length, from about 5 to about 23 nucleotides in length, from about 5 to about 22 nucleotides in length, from about 5 to about 21 nucleotides in length, from about 5 to about 20 nucleotides in length, from about 5 to about 19 nucleotides in length, from about 5 to about 18 nucleotides in length, from about 5 to about 17 nucleotides in length, from about 5 to about 16 nucleotides in length, from about 5 to about 15 nucleotides in length, from about 5 to about 14 nucleotides in length, from about 5 to about 13 nucleotides in length, from about 5 to about 12 nucleotides in length, from about 5 to about 11 nucleotides in length, from about 5 to about 10 nucleotides in length, from about 5 to about 9 nucleotides in length, from about 5 to about 8 nucleotides in length, or from about 5 to about 7 nucleotides in length.

In some instances, a barcode is from about 6 to about 25 nucleotides in length, from about 6 to about 24 nucleotides in length, from about 6 to about 23 nucleotides in length, from about 6 to about 22 nucleotides in length, from about 6 to about 21 nucleotides in length, from about 6 to about 20 nucleotides in length, from about 6 to about 19 nucleotides in length, from about 6 to about 18 nucleotides in length, from about 6 to about 17 nucleotides in length, from about 6 to about 16 nucleotides in length, from about 6 to about 15 nucleotides in length, from about 6 to about 14 nucleotides in length, from about 6 to about 13 nucleotides in length, from about 6 to about 12 nucleotides in length, from about 6 to about 11 nucleotides in length, from about 6 to about 10 nucleotides in length, from about 6 to about 9 nucleotides in length, from about 6 to about 8 nucleotides in length, or from about 3 to about 7 nucleotides in length.

### Apparatus

Automated nucleic acid sequencing apparatuses can provide a robust platform for the generation of nucleic acid sequencing reads. Unfortunately, many apparatuses have a high rate of failure, i.e., high rate of error of the sequencing reaction itself, which require manual intervention in such instances, such as re-loading of samples into flow cells. In some aspects, the disclosure provides an automated nucleic acid sequencing apparatus that requires no manual intervention in the event of a failure of a sequencing reaction. In some aspects, the disclosure provides a nucleic acid sequencing apparatus comprising: a nucleic acid library preparation compartment comprising two or more chambers configured to prepare a plurality of nucleic acids for a sequencing reaction, wherein said compartment is operatively connected to a nucleic acid sequencing chamber; a nucleic acid sequencing chamber, wherein said nucleic acid sequencing chamber comprises: (i) one or more flow cells comprising a plurality of pores configured for the passage of a nucleic acid strand, wherein said two or more flow cells are juxtaposed to one another; and an automated platform, wherein said automated platform is programmed to robotically move a sample from said nucleic acid library preparation compartment into said nucleic acid sequencing chamber

The disclosed apparatus is programmed in such a manner that said automated platform moves one or more samples from said nucleic acid library preparation compartment into said nucleic acid sequencing chamber. Upon detecting a failure of a sequencing reaction, the automated platform moves one or more samples from the failed sequencing flow cell or apparatus to the next sequencing flow cell or apparatus. In many cases, such samples comprise nucleic acid sequences that include one or more barcodes. In some cases, a plurality of mutually exclusive barcodes are added to a plurality of nucleic acids in said two or more chambers of the nucleic acid library preparation compartment, thereby providing a plurality of mutually exclusive barcoded nucleic acids within the apparatus. In some instances, the automated platform robotically moves two or more of said mutually exclusive barcoded nucleic acids into said nucleic acid sequencing chamber, in some instances by moving said mutually exclusive barcoded nucleic acids into a same flow cell of said one or more flow cells.

The present disclosure describes an apparatus for the automated detection of food-borne pathogens via the sequencing of genomic libraries from samples introduced into the instrument. In some aspects, the apparatus may comprise four main components: library chambers for library preparation, fluid handling systems, sequencing flow cells, and automation systems. Within the scope of the present disclosure, there are numerous possible uses of the pathogen detection system.

### Classification

Metadata (e.g. data ascribing a date/time to a particular strain of a pathogen) can be used to dynamically classify a sample. For example, a certain location in a food processing facility can be classified as or predicted to be: a) containing a particular pathogenic microbe, b) containing a particular serotype of a pathogenic microbe, and/or c) contaminated with at least one species/serotype of pathogenic microbe in a dynamic fashion. Many statistical classification techniques are known to those of skill in the art. In supervised learning approaches, a group of samples from two or more groups (e.g. contaminated with a pathogen and not) are analyzed with a statistical classification method. Microbe presence/absence data can be used as a classifier that differentiates between the two or more groups. A new sample can then be analyzed so that the classifier can associate the new sample with one of the two or more groups. Commonly used supervised classifiers include without limitation the neural network (multi-layer perceptron), support vector machines, k-nearest neighbours, Gaussian mixture model, Gaussian, naive Bayes, decision tree and radial basis function (RBF) classifiers. Linear classification methods include Fisher's linear discriminant, logistic regression, naive Bayes classifier, perceptron, and support vector machines (SVMs). Other classifiers for use with the invention include quadratic classifiers, k-nearest neighbor, boosting, decision trees, random forests, neural networks, pattern recognition, Bayesian networks and Hidden Markov models. One of skill will appreciate that these or other classifiers, including improvements of any of these, are contemplated within the scope of the invention.

Classification using supervised methods is generally performed by the following methodology:

In order to solve a given problem of supervised learning (e.g. learning to recognize handwriting, or a bacterial species, or a clinical condition) one has to consider various steps:

1. Gather a training set. These can include, for example, samples that are from a food or environment contaminated or not contaminated with a particular microbe, samples that are contaminated with different serotypes of the same microbe, samples that are or are not contaminated with a combination of different species and serotypes of microbes, etc. The training samples are used to “train” the classifier.

2. Determine the input “feature” representation of the learned function. The accuracy of the learned function depends on how the input object is represented. Typically, the input object is transformed into a feature vector, which contains a number of features that are descriptive of the object. The number of features should not be too large, because of the curse of dimensionality; but should be large enough to accurately predict the output. The features might include a set of bacterial species or serotypes present in a food or environmental sample derived as described herein.

3. Determine the structure of the learned function and corresponding learning algorithm. A learning algorithm is chosen, e.g., artificial neural networks, decision trees, Bayes classifiers or support vector machines. The learning algorithm is used to build the classifier.

4. Build the classifier (e.g. classification model). The learning algorithm is run on the gathered training set. Parameters of the learning algorithm may be adjusted by optimizing performance on a subset (called a validation set) of the training set, or via cross-validation. After parameter adjustment and learning, the performance of the algorithm may be measured on a test set of naive samples that is separate from the training set.

Once the classifier (e.g. classification model) is determined as described above, it can be used to classify a sample, e.g., that of food sample or environment that is being analyzed by the methods of the invention.

Unsupervised learning approaches can also be used with the invention. Clustering is an unsupervised learning approach wherein a clustering algorithm correlates a series of samples without the use the labels. The most similar samples are sorted into “clusters.” A new sample could be sorted into a cluster and thereby classified with other members that it most closely associates.

### Digital Processing Device

In some aspects, the disclosed provides quality control methods or methods to assess a risk associated with a food, with a hospital, with a clinic, or any other location where the presence of a bacterium poses a certain risk to one or more subjects. In many instances, systems, platforms, software, networks, and methods described herein include a digital processing device, or use of the same. In further embodiments, the digital processing device includes one or more hardware central processing units (CPUs), i.e., processors that carry out the device's functions, such as the automated sequencing apparatus disclosed herein or a computer system used in the analyses of a plurality of nucleic acid sequencing reads from samples derived from a food processing facility or from any other facility, such as a hospital a clinical or another. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device. In other embodiments, the digital processing device could be deployed on premise or remotely deployed in the cloud.

In accordance with the description herein, suitable digital processing devices include, by way of non-limiting examples, server computers, desktop computers, laptop computers, notebook computers, sub-notebook computers, netbook computers, netpad computers, set-top computers, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art. In many aspects, the disclosure contemplates any suitable digital processing device that can either be deployed to a food processing facility or is used within said food processing facility to process and analyze a variety of nucleic acids from a variety of samples.

In some embodiments, a digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®.

In some embodiments, a digital processing device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random-access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing-based storage. In further embodiments, the storage and/or memory device is a combination of devices such as those disclosed herein.

In some embodiments, a digital processing device includes a display to send visual information to a user. In some embodiments, the display is a cathode ray tube (CRT). In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In still further embodiments, the display is a combination of devices such as those disclosed herein.

In some embodiments, a digital processing device includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera to capture motion or visual input. In still further embodiments, the input device is a combination of devices such as those disclosed herein.

In some embodiments, a digital processing device includes a digital camera. In some embodiments, a digital camera captures digital images. In some embodiments, the digital camera is an autofocus camera. In some embodiments, a digital camera is a charge-coupled device (CCD) camera. In further embodiments, a digital camera is a CCD video camera. In other embodiments, a digital camera is a complementary metal-oxide-semiconductor (CMOS) camera. In some embodiments, a digital camera captures still images. In other embodiments, a digital camera captures video images. In various embodiments, suitable digital cameras include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, and higher megapixel cameras, including increments therein. In some embodiments, a digital camera is a standard definition camera. In other embodiments, a digital camera is an HD video camera. In further embodiments, an HD video camera captures images with at least about 1280× about 720 pixels or at least about 1920× about 1080 pixels. In some embodiments, a digital camera captures color digital images. In other embodiments, a digital camera captures grayscale digital images. In various embodiments, digital images are stored in any suitable digital image format. Suitable digital image formats include, by way of non-limiting examples, Joint Photographic Experts Group (JPEG), JPEG 2000, Exchangeable image file format (Exif), Tagged Image File Format (TIFF), RAW, Portable Network Graphics (PNG), Graphics Interchange Format (GIF), Windows® bitmap (BMP), portable pixmap (PPM), portable graymap (PGM), portable bitmap file format (PBM), and WebP. In various embodiments, digital images are stored in any suitable digital video format. Suitable digital video formats include, by way of non-limiting examples, AVI, MPEG, Apple® QuickTime®, MP4, AVCHD®, Windows Media®, DivX™, Flash Video, Ogg Theora, WebM, and RealMedia.

### Non-Transitory Computer Readable Storage Medium

In many aspects, the systems, platforms, software, networks, and methods disclosed herein include one or more non-transitory computer readable storage media encoded with a program including instructions executable by the operating system of an optionally networked digital processing device. For instance, in some aspects, the methods comprise creating data files associated with a plurality of sequencing reads from a plurality of samples associated with a food processing facility. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Program

In some embodiments, the systems, platforms, software, networks, and methods disclosed herein include at least one computer program. A computer program includes a sequence of instructions, executable in the digital processing device's CPU, written to perform a specified task. In light of the disclosure provided herein, those of skill in the art will recognize that a computer program may be written in various versions of various languages. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft®.NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CS S). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. A web application for providing a career development network for artists that allows artists to upload information and media files, in some embodiments, includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

### Mobile Application

In some embodiments, a computer program includes a mobile application provided to a mobile digital processing device. In some embodiments, the mobile application is provided to a mobile digital processing device at the time it is manufactured. In other embodiments, the mobile application is provided to a mobile digital processing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Software Modules

The systems, platforms, software, networks, and methods disclosed herein include, in various embodiments, software, server, and database modules. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### EXAMPLES

### Example 1: Detection of Transient Versus Resident Pathogens

The detection of specific pathogens serves two important roles. Firstly, it identifies the presence of important food pathogens which may have been introduced into a food handling environment but may not have been eliminated by routine sanitation practices and therefore may be passed onto other food materials being processed. Secondly, it assists in determining sources of these important pathogens that may be resident. The following protocol was used to distinguish the presence of a transient versus a resident pathogen in a food processing facility.

### Culturing and Amplification of Bacterial Nucleic Acids

First, food or environmental samples are prepared in sterile listeria culture medium (CLM) to enrich for bacteria present in the sample according to the volumes and incubation conditions in Table 1 below. Following incubation, 50 μl of each sample is transferred to a new tube and diluted with 450 μl of CL Prep Solution.

TABLE 1
Enrichment protocol for exemplary food or environmental samples
Sample Preparation

| | Sample | Volume of | |
|---|---|---|---|
| Matrix | Size | Pre-Enrichment | Incubation |
| Hot Dogs | 125 g ± 0.5 g | 1125 ± 25 | 37 ± 2° C. |
| | | mL CLM | for 26-28 h |
| Food Contact | 1 sponge | 20 ± 0.5 | 37 ± 2° C. |
| Surfaces | pre-moistened | mL CLM | for 26-28 h |
| (Stainless Steel | with 10 mL Dey- | | |
| and Plastic) | Engley Broth | | |
| Non-Food Contact | 1 sponge | 20 ± 0.5 | 38 ± 1° C. |
| Surfaces | pre-moistened | mL CLM | for 28-30 h |
| (Concrete, Rubber | with 10 mL Dey- | | |
| and Ceramic) | Engley Broth | | |

Second, 48 μl of this enriched, diluted sample is then transferred to desired wells of a 96-well plate and mixed with 2 μl sample treatment reagent capable of removing cell-free DNA. Following mixing with the sample treatment reagent, the plate is incubated in a dark location for 5 minutes at room temperature, and the plate wells are exposed to an LED light source (5000-10000 Kelvin) for 5 minutes at room temperature.

Following LED treatment, 50 μl lysis buffer is then added to each filled well of the 96 well plate, the plate is sealed, and the plate is transferred to a thermocycler for lysis at (a) 37° C. for 20 min; followed by (b) 95° C. for 10 min.

Following lysis, PCR master mix is prepared as in Table 2 below. 18 μl PCR master mix is then transferred to each well of a Clear Safety Index plate containing indexed barcode primers and the solution is mixed until the pellet in each well is dissolved, using new tips for each well.

TABLE 2
Preparation of PCR reagents

| Reagent | Per Sample | Advisory |
|---|---|---|
| PCR Master Mix | 12 μL Enzyme + | Make fresh |
| | 6 μL PCR Supplement | |

| Reagent | Per Library | Storage |
|---|---|---|
| 80% Ethanol | 800 μL absolute | Make fresh immediately before |
| | ethanol + 200 μL | library preparation |
| | molecular grade water | |

Finally, 15 μl indexed PCR master mix is then transferred to each well of a new 96 well PCR plate and mixed with 5 μl of sample from the bacterial lysis plate. The plate is then sealed with film and plated into a 96 well thermocycler to amplify and barcode the liberated bacterial DNA in a 35 cycle PCR.

### Library Preparation

Following PCR thermocycling, the 96 well plate is removed from the thermocycler and centrifuged to pool samples in each well. 5 μl of each well PCR product is transferred to an appropriate size tube to obtain a pooled product (>100 μl). 5 μl of Library Reagent 7 (which is an external control) is added to the pooled PCR product, mixed, and then 100 μl of the pooled mixed solution is transferred to a new PCR tube. 60 μl of Library reagent 9 (paramagnetic beads) is then added to this solution in the new PCR tube, and the sample is incubated at room temperature for 5 minutes. After incubation, this sample is placed into a magnetic stand and the magnetic beads from the library reagents are allowed to pellet for 2 minutes.

Following pelleting of the magnetic beads from the library reagents, the supernatant is aspirated and discarded (the supernatant volume should be approximately 160 μl). 190 μl of ethanol prepared as in Table 2 is added to the tube with the pelleted beads and removed to wash the beads. The ethanol wash is repeated once more, all of the ethanol is removed from the tube using a smaller volume pipet, and the tube is allowed to dry open at room temperature for 5 minutes. Complete removal of ethanol is verified before proceeding to the next step.

53 μl Library Reagent 8 (a suitable buffer) is then transferred to the tube with the beads and the beads are resuspended by trituration. The mixed beads are incubated at room temperature for 2 min, the beads are again pelleted in the magnetic stand, and 50 μl of the supernatant is transferred to a new tube of a PCR tube strip.

In the new PCR tube strip, 7 μl library reagent 14 (DNA end-repair buffer) is added, the sample is vortexed, 2 μl of library reagent 15 (corresponding enzyme) is added, and the sample is mixed by pipet trituration. The tube is then capped and placed in a thermocycler to run the “end prep” program (20° C. for 10 min followed by 65° C. for 5 min). After thermocycling, 60 μl of well-mixed Library Reagent 9 (paramagnetic beads) is added to the sample and the sample is mixed by trituration. The sample is allowed to incubate at room temperature for 5 minutes, and then the tube again placed in the magnetic stand to pellet the beads for 2 minutes.

Following pelleting, the supernatant is discarded (approximately 120 μl) and the beads are again washed in ethanol prepared as in Table 2 two times. After removal of all ethanol has been verified (e.g. by incubation open at room temperature for 5 minutes), the tube is removed from the magnetic stand, and 61 μl of Library Reagent 3 (molecular biology grade water) is added and the beads are resuspended by trituration. The mixed beads are incubated at room temperature for 2 minutes, and the beads are again pelleted by magnetic stand. This supernatant was retained.

### Enzymatic Treatment

60 μl of the supernatant from the bead pelleting procedure above is transferred to a new PCR tube strip, 25 ul library reagent 16, 10 μl library reagent 17, and 5 μl library reagent 20 (an adaptor mixture) are subsequently transferred to the tube, mixing after each addition. The final mixture is incubated at room temperature for 10-15 min.

Following the room temperature incubation, 60 μl library reagent 9 is added to the mixture and the sample is mixed by trituration until the mixture is homogenous without phase separation. Following a room temperature incubation for 5 minutes, the magnetic beads from this solution are pelleted on a magnetic stand for 2 minutes. The supernatant is discarded, and the tube is then removed from the magnetic stand.

To the pelleted beads, 170 μl mixed library reagent 10 (short fragment buffer) is added, and the beads are mixed with the solution by trituration. The beads are pelleted 2 minutes in a magnetic stand, the supernatant is discarded, and the beads are washed twice with library reagent 10. The beads are pelleted by magnetic stand, and all liquid solution is removed from the tube.

The pelleted beads are then mixed with 15 ul library reagent 13 (an elution buffer), and the solution is incubated at room temperature for 10 minutes.

Meanwhile, a MinION flow cell is prepared according to standard procedures, and a QC check is performed to verify at least 950 active pores are available for sequencing before proceeding.

The beads mixed with library reagent 13 are pelleted in a magnetic stand for 2 minutes, and 14.5 μl of this supernatant is collected and transferred to a new tube. 37.5 μl library reagent 12 (sequencing buffer) and 25.5 μl library reagent 11 are then added to 14.5 μl supernatant in a new tube, vortexing after each addition. This is the final library loading mix.

A priming mix is prepared by dispensing 30 μl library reagent 19 into a new tube of library reagent 18 (a flush buffer).

### Loading and Running of Flow Cell

The MinION cell prepared above is opened via its priming port, and 20-30 μl preservative buffer is removed from the priming port. 800 μl of priming mix prepared above is then dispensed into the priming port, avoiding the introduction of bubbles. The SpotON cover is discarded, and 200 ul of the Priming Mix is dispensed slowly into the priming port. Immediately before running, the final library loading mix prepared above is mixed by trituration and 75 μl of the final library loading mix is dispensed onto the Spot-ON port of the MinION cell, dispensing dropwise carefully to avoid the introduction of bubbles. The MinION device lid is closed, and the sequencing reaction is executed via software on the computer connection of the MinION device according to standard procedures.

### Example 2: Kits for Detection of Transient Versus Resident Pathogens

### Kit Components

In some embodiments, a kit of the disclosure can comprise one or more of the items described below:

| System | Storage | Reagent Kit |
|---|---|---|
| Component | (° C.) | Number |
| Library Reagent 3 [molecular | −18 to −22 | I |
| biology grade water] | | |
| Library Reagent 7 [external control] | | |
| | −18 to −22 | I |
| Library Reagent 8 [buffer] | −18 to −22 | I |
| Library Reagent 9 [Paramagnetic beads] | 2 to 8 | III |
| Library Reagent 10 [Short fragment buffer] | −18 to −22 | I |
| Library Reagent 11 [Library loading beads] | −18 to −22 | I |
| Library Reagent 12 [sequencing buffer] | −18 to −22 | I |
| Library Reagent 13 [elution buffer] | −18 to −22 | I |
| Library Reagent 14 [DNA end-repair buffer] | −18 to −22 | I |
| Library Reagent 15 | −18 to −22 | I |
| Library Reagent 16 | −18 to −22 | I |
| Library Reagent 17 | −18 to −22 | I |
| Library Reagent 18 [flush buffer] | −18 to −22 | I |
| Library Reagent 19 | −18 to −22 | I |
| Library Reagent 20 [adaptor mixture] | −18 to −22 | I |
| Sample Treatment | −18 to −22 | II |
| Lysis Buffer | −18 to −22 | II |
| Enzyme | −18 to −22 | II |
| PCR Supplement | −18 to −22 | II |
| Clear Salmonella Index Plates | Ambient | II |
| CLM Media | Ambient | Shipped |
| | | Directly |
| CL Prep Solution | Ambient | Shipped |
| | | Directly |
| MinION Flow Cell, R9.4.1 | 2 to 8 | Shipped |
| | | Directly |
| Minion Sequencer | N/A | Shipped |
| | | Directly |
| Thermal Cycler | N/A | Shipped |
| | | Directly |
| Light Table | N/A | Shipped |
| | | Directly |
| 96-Well Magnetic Ring Plate | N/A | Shipped |
| | | Directly |
| 96-Plate Well Plates | N/A | Shipped |
| | | Directly |

In some embodiments, a kit of the disclosure can comprise one or more of the items described below:

### Shelf Life and Storage of Kit Components

Reagents in the current kit configuration are divided as follows: Reagent Kit I, Reagent Kit II, Reagent Kit III. The Reagent Kit I and III have an expiration date of 3 months after manufacturing date. The Reagent Kit II has an expiration of 9 months after manufacturing date. The expiration dates are valid so long as the kits are kept at their respective storage conditions.

The ALPAQUA Magnum FLX magnet plate contains strong neodymium magnets. Individuals with pacemakers or implantable cardioverter defibrillators should avoid contact with this component. Keep this component away from metal objects, other magnets, electronic equipment like computers, digital media devices (for example USB drives and mobile telephones), and other media with embedded chips (such as credit cards and passports)—proximity to this component can corrupt the data on these devices.

Recommendations for Kit Use

Clean work stations both before and after use with a fresh 5000 ppm hypochlorite solution (approximately 1:10 dilution of household bleach or 1:16 of 8.25% hypochlorite industrial bleach). Bleach is recommended because it can both disinfect and degrade nucleic acids on surfaces, both of which are potential sources of contamination. If the use of bleach is not desirable, it is recommended to use compatible products that still accomplish both of these goals. For example, a two-phase wipe down with quaternary ammonia and a product like “DNA Away” (Molecular Bio-Products, San Diego, Calif.).

### Example 3: Methods for Detection of Transient Versus Resident Pathogens

Media and Supplement Preparation:

Suspend 53.8 g of Clear Listeria Medium (CLM) in 1 L of deionized water.

Mix thoroughly.

Heat as needed to dissolve completely.

Autoclave at 121° C. for 15 minutes.

Post-enrichment Sample Preparation

Matrix Enrichment Guide, prepare samples for enrichment, using the respective media volume, incubation time, and incubation temperature.

Following enrichment, remove 50 μL of enriched sample, and combine with 450 μL of CL Prep Solution. Once completed for all samples, take through to Sample Preparation.

TABLE
Matrix Enrichment Guide Sample Preparation

| | Sample | Volume of | |
|---|---|---|---|
| Matrix | Size | Pre-Enrichment | Incubation |
| Hot Dogs | 125 g ± 0.5 g | 1125 ± 25 | 37 ± 2° C. |
| | | mL CLM | for 26-28 h |
| Food Contact | 1 sponge | 20 ± 0.5 | 37 ± 2° C. |
| Surfaces | pre-moistened | mL CLM | for 26-28 h |
| (Stainless Steel | with 10 mL Dey- | | |
| and Plastic) | Engley Broth | | |
| Non-Food Contact | 1 sponge | 20 ± 0.5 | 38 ± 1° C. |
| Surfaces | pre-moistened | mL CLM | for 28-30 h |
| (Concrete, Rubber | with 10 mL Dey- | | |
| and Ceramic) | Engley Broth | | |

A. Sample Sheet Generation

On the laptop connected to the MinION sequencer, open the “Samplesheet TEMPLATE” on the desktop to open an Excel sheet, containing two sheets, one titled “Template” and another titled “Example_samplesheet.”

On the top left of the page, click on “File,” then “Save a Copy . . . .”

Rename the document title using the following format:
mmddyy_ExperimentName_FlowCell_ID
For example: 011719_AOAC_batch_1_FAH46157

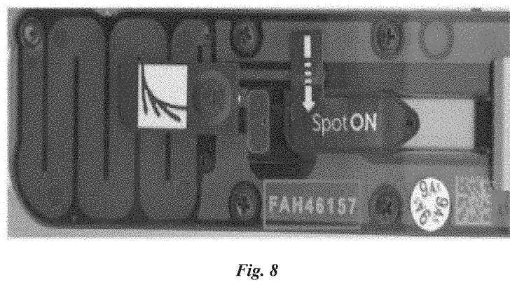

In order to obtain a Flow Cell ID, retrieve a new flow cell from the 2-8° C. storage. Note the Flow Cell ID of the flow cell (found on the top face of the flow cell, in yellow lettering, FIG. 8) and return the flow cell back to the 2-8° C. storage. This particular flow cell will be later. Close the template Excel sheet and open the newly copied Excel sheet. Fill out the “Template” sheet with the sequencing run information, sample information, and sample location on a 96-well plate. Note that a “*” indicates a required field. The “Example_samplesheet” tab can be a reference guide to completing the samplesheet.

The definitions of the samplesheet's required information are as follows:

“MinION I” is located on the sequencer itself

“Sample ID” is the name created in Step 4, and is also the title of the samplesheet;

“Flow Cell ID” is found on the flow cell in yellow lettering

“Number of Samples in Run” states (and should match) how many samples are being processed in this test run. The minimum number of samples for a test run is 32.

“Sample_Name” is the description of a sample in a given sample well.

Save the samplesheet and transmit the document electronically to Reagent Preparation

| Reagent | Per Sample | Advisory |
|---|---|---|
| PCR Master Mix | 12 μL Enzyme + | Make fresh |
| | 6 μL PCR Supplement | |

| Reagent | Per Library | Storage |
|---|---|---|
| 80% Ethanol | 800 μL absolute | Make fresh immediately before |
| | ethanol + 200 μL | library preparation |
| | molecular grade water | |

Sample Preparation

NOTE: The instructions below assume the use of a full 96-well plate; if preparing partial or multiple plates, adjust reagent placements and volumes according to the fraction of the plate being used. Remove the Lysis Buffer and the amber Sample Treatment tube from −20° C. and let thaw.

Pipette mix enriched samples (combined with CL Prep Solution, as per Table A) and ensure there is no phase separation. Using the Sample Sheet submitted as a guide, pipette 48 μL of enriched, diluted sample into individual wells of the Sample Preparation Plate (96-well plate).

NOTE: Sample Treatment is extremely light-sensitive; protect Sample Treatment-loaded plates/tubes from light. Protect the stock reagent tube by working efficiently (multichannel and reservoir use).

Vortex and add 2 μL of Sample Treatment reagent to each well of the Sample Preparation Plate. Pipette mix 5-10 times.

NOTE: Ensure QC so that each sample well receives Sample Treatment reagent. Change pipette tips after dispensing. Also ensure that the 2 μL is being pipette mixed into solution, and not in air bubbles.

a) If using a multichannel pipette, aliquot 25 μL of Sample Treatment Working Stock into an 8-tube strip. Arrange the tubes into a single column in a rack and use as you would a reagent reservoir. This can also be done with a spare PCR plate.

1. Let the plate incubate in a dark location (foil or deep shade) for 5 min at room temperature.

Turn on the provided Light Table, place the plate onto the lit surface, and allow it to sit for 5 min at room temperature.

3. Retrieve the samples from the Sample Treatment step that are ready to be lysed. Add 50 μL of Lysis Buffer to each sample-containing well. Pipette mix 3-5 times.

4. Seal the Sample Preparation Plate with sealing film and place in the provided thermal cycler. Run the program “Lysis.”

5. Seal the Sample Preparation Plate with sealing film and place in the provided thermal cycler. Run the program “Lysis.”
Remove Enzyme and PCR Supplement from −20° C. and let thaw.

PCR

Prepare the PCR master mix.

Add 18 μL of freshly prepared PCR Master Mix to each well of a Clear Safety Index Plate. It is critical to use a new tip for each well; never reuse a tip that has been used to resuspend or transfer the mix.

Gently pipette up and down 10 times until the reagent pellet dissolves. Avoid making bubbles. Change the pipette to 15 μL and pipette mix again 10 times.

Transfer 15 μl from the well(s) of the Clear Safety Index Plate to the respective well(s) of a new 96-well PCR plate. Ensure orientation and destination.

Remove samples from Lysis program and add 5 μl of each sample from the Sample Preparation Plate to the respective wells of the PCR plate. Pipette mix the sample into the solution, approximately 5-10 times.

NOTE: For sample tracking, it is critical that the identity of each sample can be traced to its respective well on the Clear Safety Index Plate. If a positional error does occur at this stage, note the new position of sample in the Sample Sheet—analysis results will ultimately be linked to the samples' position on the Clear Safety Master Mix Plate.
Seal the PCR plate with sealing film and place in the provided thermal cycler. Run the program “PCR.”

Library Preparation

NOTE: All PCR plates that are planned to be sequenced in one run will be pooled together to prepare one pooled sequencing library.

NOTE: The 200 μL aliquot of Library Reagent 9 must be warmed to room temperature before use. It is also important to vortex immediately prior to use. Library Reagent 9 can form highly viscous clusters at the bottom of the tube that can only be effectively suspended by vortexing.

NOTE: Library Reagent 15, 17, 19, and 20 all contain proteins that are sensitive to temperature changes. Only remove these reagents from −20° C. storage immediately prior to use and return back to −20° C. storage after use.

NOTE: All reagents, before use, should be spun down in a microcentrifuge and pipette-mixed OR vortexed where stated.
Remove a tube of Library Reagent 9 from 4° C. storage and allow it to reach room temperature (approx. 10 min); also remove a tube of Library Reagent 7 and 8 from −20° C. and let thaw.

Remove PCR Plate from the thermal cycler and spin briefly in a benchtop plate centrifuge. Remove the sealing film.

From the PCR Plate, pool 5 μL of each sample's PCR product in an appropriately-sized tube to obtain at least 100 μL of pooled product. An 8-tube strip may be used as an intermediate to expedite pooling.

Add 5 μL of Library Reagent 7 to the PCR pool.

Briefly vortex pooled PCR product and pipette 100 μL into a tube of an 8-tube strip. Set aside the original tube—subsequent steps will work out of this tube strip.

Vortex the aliquot of Library Reagent 9 for 5-10 sec and ensure it's well homogenized. Immediately add 60 μL to the pooled PCR product. Set the pipette to 130 μL and mix thoroughly by pipetting up and down approximately 10 times. Ensure color of the mixture is homogeneous and there is no phase separation.

Incubate at room temperature for 5 min.

Place the tube strip containing the mixture into the magnetic stand and leave for 2 minutes, allowing to pellet in a ring leaving a clear supernatant.

NOTE: Do not remove the tube from the magnetic stand unless instructed.

With the tube strip still in the stand, use a p200 pipette and place the tip at the bottom center of the tube. Aspirate slowly to avoid disturbing the ring and discard the supernatant (approximately 160 μL).

Add 190 μL of freshly prepared 80% ethanol. Aspirate fully and discard the supernatant.

Repeat step 10 once more for a total of 2 ethanol washes. After discarding the second wash, use a p20 pipette to remove any remaining ethanol without disturbing the pellet.

Let the sample dry for 5 min at room temperature, or until no visible ethanol remains. Visually inspect to ensure there is no ethanol remaining in the tube. Do not proceed until all drops of ethanol have evaporated and the sample well is completely dry.

Remove a tube of Library Reagent 14 and Library Reagent 15 from −20° C. and let thaw.

Once completely dry, remove the tube strip from the magnetic stand.

Pipette 53 μL of Library Reagent 8 into the well containing the pellet and resuspend. Mix thoroughly by gently pipetting up and down approximately 10 times until the solution appears homogeneous.

Incubate at room temperature for 2 min (not in the magnetic stand).

Move the tube strip to the magnetic stand and incubate at room temperature for 2 min to allow to pellet.

Transfer 50 μL of the supernatant to a new well of the tube strip. Remove the tube from the magnetic stand. To this new well, add:

NOTE: Do not vortex the Library Reagent 15, as it may result in protein damage. Spin the tube in a microcentrifuge and pipette gently to ensure homogeneity.

Reagent Volume

Library Reagent 14 (vortex) 7 μL

Library Reagent 15 (pipette mix) 3 μL

Set the pipette to 45 μL and mix well by pipetting up and down approximately 10 times.

Cap the 8-tube strip and place in the provided thermal cycler. Run Program “End Prep”

Retrieve the tube strip from the thermal cycler.

To the end-prepped well, add 60 μL of well-vortexed Library Reagent 9. Set the pipette to 90 μL and mix well by pipetting up and down approximately 10 times

Incubate at room temperature for 5 min.

Place the tube strip containing the sample/bead mixture into the magnetic stand and leave for 2 minutes, allowing to pellet in a ring leaving a clear supernatant.

NOTE: Do not remove the tube from the magnetic stand unless instructed.

With the tube strip still in the stand, use a p200 pipette and place the tip at the bottom center of the tube. Aspirate slowly to avoid disturbing the ring and discard the supernatant (approximately 120 μL).

Add 190 μL of freshly prepared 80% ethanol. Aspirate fully and discard the supernatant.

Repeat step 25 once more for a total of 2 ethanol washes. After discarding the second wash, use a p20 pipette to remove any remaining ethanol without disturbing the pellet.

Let the sample dry for 5 min at room temperature, or until no visible ethanol remains. Visually inspect to ensure there is no ethanol remaining in the tube. Do not proceed until all drops of ethanol have evaporated and the sample well is completely dry.

Once completely dry, remove the tube strip from the magnetic stand.

Pipette 61 μL of Library Reagent 3 into the well and resuspend. Mix thoroughly by gently pipetting up and down approximately 10 times until the solution appears homogeneous.

Incubate at room temperature for 2 min (not in the magnetic stand).

Move the tube strip to the magnetic stand and incubate at room temperature for 2 min to allow to pellet.

Transfer 60 μL of the supernatant to a new well of an 8-tube strip. Remove the tube strip from the magnetic stand. To this well, add:

Note: Do not vortex Library Reagent 17 OR Library Reagent 20, as it can lead to protein damage. Spin the tubes in a microcentrifuge and pipette-mix to ensure homogeneity.

| Reagent | Volume |
|---|---|
| Library Reagent 16 (pipette mix) | 25 μL |
| Library Reagent 17 (pipette mix) | 10 μL |
| Library Reagent 20 (pipette mix) | 5 μL |

Note: Library Reagent 16 and Library Reagent 17 are viscous due to the high glycerol content. Pipette volume slowly to ensure the pipetting of an accurate volume.

1. Set the pipette to 80 μL and mix by gently pipetting up and down approximately 20 times.

2. Incubate at room temperature for 10-15 min. Remove a tube of Library Reagent 10, 11, 12, 13, 18, and 19 from −20° C. and let thaw.

Vortex Library Reagent 9 tube briefly to homogenize and immediately add 60 μL. Mix thoroughly by pipetting up and down approximately 10 times. Ensure color of the mixture is homogeneous and there is no phase separation.

Incubate at room temperature for 5 min.

Place the tube strip in a magnetic stand for 2 min and allow to pellet in a ring, leaving a clear supernatant.

NOTE: Do not remove the tube strip from the magnetic stand unless instructed.

Using a p200 pipette, place the tip at the bottom center of the tube. Aspirate slowly to avoid disturbing the ring and discard the supernatant (approximately 160 μL).

Remove the tube strip from the magnetic stand and pipette 170 μL of vortexed Library Reagent 10 onto the ring attached to the wall and bring into solution by continually aspirating and dispensing onto the wall near the ring. Afterward, mix by gently pipetting up and down approximately 10 times to ensure solution is homogeneous.

Return the tube strip to the magnetic stand for approximately 2 min, and allow to pellet in a ring, leaving a clear supernatant.

Using a p200 pipette, place the tip at the bottom center of the tube. Aspirate slowly to avoid disturbing the ring and discard the supernatant.

Repeat steps 39-41 for a total of two washes with the Library Reagent 10.

Using a p20 pipette, remove any remaining volume from the well.

Remove the tube strip from the magnetic stand and add 15 μL of Library Reagent 13 onto the ring attached to the wall and bring into solution by continually aspirating and dispensing onto the wall near the ring. Afterward, mix by gently pipetting up and down approximately 10 times to ensure solution is homogeneous.

Incubate at room temperature for 10 min.

### Example 4:MinION Flow Cell Quality Check

A flow cell must be Quality Checked before it is used for sequencing. To perform the QC check:

Turn on the laptop connected to the MinION sequencer, and login

If the MinION sequencer is not yet plugged in, connect it to the laptop using any one of the available USB ports. Ensure there is no flow cell currently inserted into the device. Once a flow cell has passed the Quality Control check, it is ready for use.

### Example 5: Final Loading Mix and Priming Mix

Move the tube strip to a magnetic stand and allow to pellet for approximately 2 min.

Using a p20 pipette, place the tip at the bottom center of the tube. Slowly aspirate 14.5 μL of supernatant and transfer to a new 1.5 mL tube.

To this new tube, add:
Note: Ensure the Library Reagent 11 is mixed well via pipette mixing immediately prior to taking an aliquot. The beads in this solution can settle quickly.

| Reagent | Volume |
|---|---|
| Library Reagent 12 (vortex) | 37.5 μL |
| Library Reagent 11 | 25.5 μL |
| (vortex briefly and then pipette | |
| mix) | |

This is the Final Library Loading Mix.

Note: Do not vortex the Library Reagent 19, as it can lead to protein damage. Also do not vortex the tube of Library Reagent 18 after the Library Reagent 19 has been added to it.

Prepare the Priming Mix by dispensing 30 μL of Library Reagent 19 into a new tube of Library Reagent 18.

This is the Priming Mix.

MinION Flow Cell Loading

Obtain a MinION Flow Cell that has passed Quality Check.

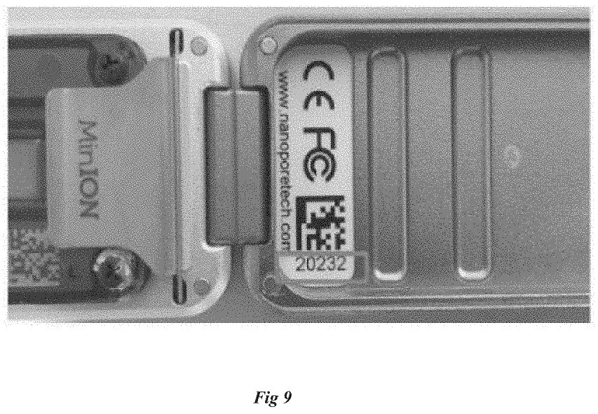

Gently slide open the priming port of the Flow Cell. Using a p1000 pipette, slowly take out approximately 20-30 μL of the preservative buffer (FIG. 9).

NOTE: The volume must not be removed by pressing down the pipette plunger. It should only be done by turning the plunger anti-clockwise until a small volume of preservative buffer is removed.

Discard the aspirated preservative buffer and tip.

Use a p1000 pipette to pipette-mix the Priming Mix and aspirate 800 μL. Position the pipette tip absolutely vertically and settle the tip firmly into the priming port. Slowly dispense the 800 μL of Priming Mix into the Priming Port.

Faulty flow cell priming can significantly lower the success rate of a sequencing run. To prevent this, consider the following:

a) Pipette slowly and steadily. AVOID ACCIDENTALLY ASPIRATING DURING PRIMING: the priming step serves to push a preservative solution away from the sensor array—aspiration can cause it to instead mix with the Priming Mix.

b) Leave a small volume of Priming Mix in the pipette tip at the end of dispensation in order to avoid introducing any air bubbles when dispensing the Priming Mix. That is, there should be a small amount of Priming Mix still in the tip at the end.

c)Before releasing the pipette plunger, remove the pipette tip completely from the priming port. Releasing the plunger while removing the tip can cause accidental aspiration.

Note: Ensure the internal fluids move through the channel as the Priming Mix is dispensed into the port.

Note: Perform Steps 7-8 immediately after Step 6. The time differential between Step 5 and 6-7 must be less than 2 minutes.

Gently lift open the plastic SpotON sample port cover and discard.

Very slowly (and without aspiration) dispense 200 μL of the Priming Mix into the Priming Port using slow, steady pressure. Pipetting too quickly will cause fluid leakage out of the SpotON port; observing the Spot-on port for the appearance of rising fluid can help you gauge your pipette speed.

Immediately before loading, mix the final library loading mix (previously prepared) thoroughly by pipetting up and down approximately 10 times to ensure the solution is homogenous. Ensure no bubbles are formed due to hasty pipette-mixing, as the transfer of these bubbles into the flow cell can compromise the sequencing run.

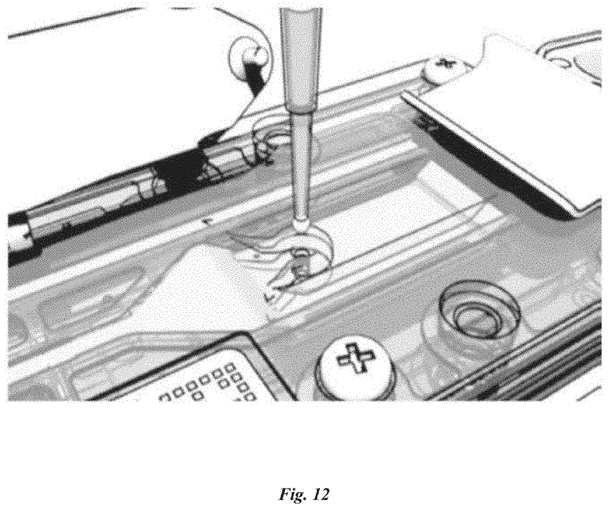

Dispense 75 μL of the final library loading mix onto the SpotON port of a prepared MinION Flow Cell. Dispense dropwise, ensuring each drop fully enters the port prior to dispensing another drop. This can be accomplished by gently touching the droplet—but not the pipette tip—to the SpotON port (see FIG. 12).

NOTE: Do not dispense final library loading mix directly into the SpotON port. Instead, position the pipette tip above the open SpotON port and introduce droplets of the final library loading mix by either having the formed droplets drop onto the open port or introducing the formed droplet to the air-liquid interface inside the open SpotON port. Near the end of the dispensation, only introduce droplets that do not contain air, as an air bubble can compromise the sequencing run.

Close the lid of MinION device.

On the sequencer laptop, the GridION program should be at the main page for starting a new sequencing run.

Ensure that the flow cell that was Quality Control checked is still docked on the MinION.

Select the flow cell, and then on the bottom of the page, select “New Experiment.” A pop-up box should appear.

Enter the title of the submitted samplesheet as the “Experiment”. The name must be exactly the same for successful analysis. Leave the remaining fields at default options.

Select “Kit” the left side of the pop-up box and select the “SQK-LSK-109” kit.

On the “Basecalling” tab, turn off Basecalling.

On the “Run Options” tab, change the length of sequencing from 48 hours to 4 hours. Leave the remaining fields at default settings.

Skip the “Output” and “Custom Script” tabs.

Select “Start Run” to begin sequencing.

Data Analysis and Interpretation

Email notification will be sent out to the operator when result of analysis is available.

### Example 6: Monitoring of a Poultry Supply Chain for Salmonella Infection

The computer-implemented sequencing-based tracking methods described herein (“Clear Safety”) are used to monitor Salmonella prevalence, quantity and identity at various sampling points along the supply chain in a poultry establishment. The poultry supply chain typically consists of the following: Feed Providers, Breeding Stock, Pullet Farm, Breeder Farm, Hatchery, Broiler Farm, and Processing. In the United States, the FDA-recommended regulatory actions depend on the serovar of Salmonella found and the animal species that receives the feed. For poultry feed, the U.S. government requires that it be absent of S. Gallinarum and S. Enteritidis.

In one example, a computer-implemented method is used for monitoring and evaluating genetic similarities between pathogen strains in a given supply chain by sampling a series of locations at varying times. In a first step, a computer-based method is used to sample a given location at a given point in time to acquire nucleic acid sequence information from a given pathogen strain, and a metadata resource is created for the test sample including data points and dimensions such as time and location. In a second step, the computer-based method is used to sample the same location at a different time or a different location at the same time to acquire nucleic acid sequence information relevant to the presence of a second pathogen strain, and metadata for the second test sample based on the data points (time and location) is applied to the sequence information. Next, a module is applied for computing genetic distances between the acquired nucleic acid sequences of the first and the second pathogen strains. In one example, if the first pathogen strain and the second pathogen strain are identified as the same strain, then a source location of the pathogen strains contamination is created based on the stored metadata information (including sampling time, sampling location etc.)

During the processing of an animal carcass into animal meat or collection of products from animals (e.g. eggs) there can be several chemical and mechanical control points assessed for pathogen contamination to reduce the level of Salmonella on the carcass. Using the computer-based method above, identifying the serotype and load after each control point can inform the establishment how effective those control points are over time. For example, in the case of processing chicken pullets into end chicken cuts, the “locations” described above monitored by the computer-implemented method can comprise steps and locations in the animal processing scheme such as reception of the animals (e.g. animal cages and/or feed), slaughter of the animals (e.g. animal carcasses after de-feathering, evisceration, and/or pre-chilling), processing of the carcasses (e.g. knives, cutting boards, or operator hands), or ending cuts (e.g. processed leg, wing, and/or breast meat). This information can be used to identify trends (i.e., indicate when the process is going out of control) and therefore illustrate the risk an establishment is taking when releasing product into commerce or preparing for another production cycle. To take another example, in the case of egg production, the “locations” described above monitored by the computer-implemented method can comprise steps and locations in the egg production scheme such as rearing (e.g. paper on the production floor, cage racks, and/or feed), egg production (e.g. hens themselves or dust, floor, nest box, and egg belt of the egg production shed), or grading (e.g. egg grading floor).

Depending on the test results and the sampling point within the supply chain, the user may take different actions. For example, some farms will test their feed to see if the serovars in their feed are being passed from foodstuffs to their pullets, processed chickens, or graded eggs. The need to test feed will vary from supplier to supplier and from country to country.

In the case of egg production, one example of critical point in the supply chain involves laying hens. Fecal contamination of eggshells during oviposition can result in the exposure of hatching chicks to Salmonella. Some serotypes, notably S. Enteritidis and S. Heidelberg, can colonize the reproductive tissues of hens and are deposited inside the eggs, causing infection of chicks. Consequently, some companies choose to monitor the serovars present in their breeder farms and in their hatcheries to see if certain serovars are being transmitted vertically. The detection of certain serotypes at this stage can impact the disposition of those eggs.

Another example involves the broiler farms where chickens are raised until slaughter. The “houses” containing these chickens are sampled to understand the identify of Salmonella present as well as the quantity. If certain serotypes are detected, or if high quantities of salmonella are detected, the establishment may choose to destroy the flock within that house or process the flock in a manner that minimizes exposure to other flocks.

Using the computer-based method above, establishments can 1) identify the type and level of Salmonella in a sample, 2) view where said Salmonella was detected on a digital floorplan as well as a representation of the supply chain in the Clear View software, 3) determine if said pathogen has been detected previously, and if so, when and where, and 4) identify other functional characteristics of that organism, such as antimicrobial resistance, heat tolerance, or clinical relevance. Coupled with other metadata, Clear Safety can present the user with a “risk score” that is dependent on parameters they set for themselves, i.e., the identity of Salmonella in the sample, the level of Salmonella in the sample, the functional genetics (i.e., antibiotic resistance or pathogenicity), and when/where in the supply chain it was detected. Such information can be used to understand the nature, source, and level of risk the establishment is taking when determining product disposition and can inform their mitigation strategies throughout the supply chain.

### Example 7: Monitoring of Pathogen Strains by a Ready-to-Eat Food Manufacturer

A food manufacturer monitors their manufacturing environment for microbial pathogens through sampling. With the computer-implemented pathogen tracking systems and methods herein (“Clear Safety”), the manufacturer is able to 1) identify the pathogen in the sample, 2) view where the pathogen was detected on a digital floorplan in the software (“Clear View”), 3) determine if said pathogen has been detected previously, and if so, when and where, and 4) identify other functional characteristics of that organism, such as antimicrobial resistance, heat tolerance, or clinical relevance.

Through machine learning, Clear Safety will use result metadata to design sampling plans and investigations tailored for the specific pathogen of interest. For example, if a recurring strain of pathogen is detected six months after it was last detected, the system will automatically create an investigative sampling plan for the manufacturer that includes sites where the strain was previously detected as well as “vector sites” that are chosen to ascertain the extent and potential source of the contamination. Such a sampling pan can be generated, in some instances, by applying a non-linear algorithm to a time series of location contamination data, or a time series of apparent pathogen introduction locations to extract the most common contaminated locations or pathogen introduction locations. Such time location contamination data can also incorporate data such as employee traffic patterns, water presence, and processing facility load to determine if sampling should be updated according to cyclical or random changes in employee, starting material, or product throughput.

Similarly, a similar algorithmic scheme can be applied to implement root cause analysis by applying a machine learning algorithm to a data set comprising time series of e.g. pathogen introduction locations, the corrective action that was taken for the incidents, and whether the contamination was resolved or not to suggest to the product manufacturer/processor what a potential root cause and corrective action can be implemented for the current investigation.

The data can be compiled in a way that can be easily viewed and understood by anyone (including auditors and federal investigators) as documentation of these incidents as well as the follow-up activities (hazard mitigation) are required by law.

Through Environmental Mapping with Clear Safety, the user can view contamination incidents on floorplans over time and view genetic commonalities between contaminants. For example, the user can see the movement of a specific strain of Listeria through the manufacturing environment over time and, when coupled with other metadata including employee traffic patterns, water presence, and food product flow, the manufacturer can ascertain the source of the contamination and potentially predict other points of contamination. This allows them to identify the true source of the contamination and prevent it for recurring.

Through profiling (e.g., identifying functional characteristics from the pathogen's genome), the system can prescribe to the manufacturer mitigation activities tailored to the specific incident. For example, the system may identify known markers (e.g. involving qacEΔ1 or qacF which impart resistance to quatemary ammonium sanitizers, or pcoR, pcoC, and pcoA which impart resistance to naturally antimicrobial copper surfaces) that impart the organism with increased resistance to a particular sanitizer or staying power on surfaces, and the system would accordingly recommend a specific sanitizer to use (e.g., oxidizing sanitizers instead of quaternary ammonium ones, or application of additional sanitization procedures to copper surfaces). Additionally, the system may recognize the strain as one that has been implicated in clinical cases; this information could impact how the manufacturer assesses the risk of that incident and the extent of precautions they will take going forward.

Coupled with other test data, i.e., microbiome or non-pathogenic indicator organisms, Clear Safety can monitor the prevalence and quantity of various organisms detected in samples from the food and food manufacturing environment. Through statistical process control monitoring, the system can recognize and report to the user when the food safety system is out of control, i.e., results are trending upward or patterns are identified that correlate to an impending problem or contamination event. For example, indicator organism (non-pathogenic) detection and quantification can be used to ascertain how sanitary a site or object may be over time; a consistently unsanitary site suggests that hygiene measures are inadequate and presents an increased risk of harboring a pathogen. Such information can be used to “predict” when a manufacturer may encounter a pathogen.

Over time, aggregated data from Clear Safety users can be mined to better understand the dynamics of environmental contamination across various food products and manufacturing practices. Such information provides an academic assessment of the nature and dynamics of food contamination and present valuable insights to industry, academia, and government.

### Example 8: Establishment of a Pattern Tracking Feature for Pathogen Detection and Reporting

An instrument for tracking and detection of resident or transient pathogens in test samples is presented. The pattern tracking relies on several data points and dimensions collected from test samples.

The analytical process begins with an instrument specialized for sample processing called “Skybox”. In this instrument, samples are processed using reagents, kits and hardware devices that are designed to extract raw genetic sequence data from test samples. Once the genetic data from test samples is obtained, the sequence data is fed into a data base called BIP (Bio Pipeline).

The BioPipeline database is generated up front for use by stacking multiple static databases (read-only). For example, the BIP-DB consists of a Whole-genome sequence Pathogen-Database (comprising sequences of all the pathogen genomes that are desired to be detected/analyzed) as a foundational database. From the Whole-genome-sequence Pathogen database, alleles are extracted to create an allele BLAST database (P-AB-DB) and a substring vector database (SUB-VDB). The substring vector database comprises k-mer natural vectors corresponding to each characteristic allele. As the next step, genetic distance groups are created based on Single Nucleotide Polymorphism (SNP) distance from the Whole-genome-sequence Pathogen database (P-WGS-DB). A genetic distance vector database DB (GD-VDB) is then generated based on the SUB-VDB. Sequences obtained by genetic testing of samples are classified by alignment (based on genetic distance) into alleles using the P-AB-DB database. The test samples are compared to the database to identify positive cases of pathogen detection (S_pos). The S_pos IDs are assigned to a PT_ID using genetic distance vector database (GD-VDB) and the substring vector database (SUB-VDB). Next, the data from the BioPipeline database is fed into an AIR dynamic analytical system. The Analytical system uses the detected pathogens, the groups, as well as aggregated Time and Location dimensions (obtained from the sampling meta-data information) and other sources to provide business insights and predictions. Specifically, the AIR analytical system aggregates positive sample detections, together with Time and Location information into a database (DTL-DB). Next, the Aggregate Positive Sample Groupings (PT_ID) are aggregated into a Database (PT-DB). Following this, analytics are run on the DTl-DB, PT-DB and other databases to extract insights, such as transient-vs resident risks or outbrake flows and stored in the database (AIR-DB).

Following this, the data from the AIR analytical system is fed into the APP application system, where business insights, predictions, and prescriptions are displayed or further filtered in the Application.

### Example 9: Generation of a Computer-Based Web Application for Pathogen Detection

In this example, a web application for management of pathogen samples, reporting of pathogen detection and business insights is described.

The process of pathogen detection and reporting comprises several steps starting with sample collection from different time points or locations, followed by storage of additional parameters as metadata during the next sample registration step.

Following this, the sample is prepared for testing, where the one or more samples are loaded into flow cells placed on indexed plates that are part of the Clear Safety Instrument. The Clear Safety instrument is a device that is installed at a given customer location and includes a robotic system (such as a liquid handler) and DNA sequencer (e.g. GridION from Oxford Nanopore Technology), as well as various peripherals. The robotic system in the Clear Safety Instrument is controlled by a software tool called the Venus Software (a Hamilton company software which is integrated with the Clear Safety Instrument). Sequencing reagents are added to the flow cells in the Clear Safety Instrument to perform a quality check, wherein the Venus computer software is used to control the robotic instrument equipped for sample processing. The robotic instrument processes samples using automated liquid handling procedures and nanopore sequencing procedures to obtain genetic sequencing information from the samples. The genetic sequence data is then uploaded by the robotic instrument to the Clear Labs Cloud where the subsequent steps of analysis and reporting of the sequencing steps are performed. Clear Labs Cloud is a software platform running on Google Cloud (GCP) providing data analysis, monitoring and applications support. The analytical reports are then fed into a web application called Clear View where the genetic sequencing data is mined for the multi-dimensional metadata information stored during sample acquisition and processing together with environmental mapping to produce business insights. The Clear view web application is equipped to produce insights on user management, floor plan management, product management, client management etc.

The Clear Safety Instrument is placed under the control of the Customer Network. The data from the Clear Safety Instrument then passes through the Customer router/Firewall. The Clear Safety Instrument communicates with the Clear Labs Cloud via Internet, using the protocols and ports that are outlined in the diagram. The Clear Labs Cloud, is in turn a software platform, running on Google Cloud (GCP), providing support for data analysis, monitoring, and applications. The data from the Clear Labs Cloud is then fed into the Clear View Web Application for sample management, reporting the analytical results to the customer and using the stored sample metadata to extract business insights related to user, floorplan, product or client management.

### Example 10: Generation of a Computer-Based Method of Pathogen Detection and Tracking

Building a pattern tracking (Resident/Transient Pathogen Detection) is a computer-based feature that relies on several data points and dimensions collected from test samples. Examples of such features include time and location of pathogen detection and genetic similarity between the detected pathogen strains. In this feature, a specific sample is collected at a specific time, which is stored in metadata associated with the sequence of any pathogen strains detected. The specific location where the sample was collected is also stored a metadata dimension. Genetic distance, calculated as the indirect single-nucleotide polymorphism among the samples testing positive, determined by pre-calculated groups is then calculated. The genetic distance between pre-calculated groups is taken as an indicator of whether two pathogens are an identical strain or not (low genetic distance being an indicator they are identical), which in turn is an indicator the strain is resident. Geographical flows between detected locations determined by this process can be used as an indirect measure of how similar pathogens (residents) can travel along certain locations over a period of time.

### EMBODIMENTS

The following embodiments are provided by way of example only and are not intended to be limiting in any way.
Embodiment 1. A computer-implemented method of monitoring a pathogen strain, comprising,
(a) associating, at a computer:
   (i) nucleic acid sequence information from said pathogen strain;
   (ii) metadata identifying a first sampling location for said nucleic acid sequence information from said pathogen strain; and
   (iii) metadata identifying a first sampling time for said nucleic acid sequence information from said pathogen strain;
(b) maintaining, in media accessible by said computer, a module for computing genetic distances between at least two nucleic acid sequences;
(c) associating, at said computer:
   (i) nucleic acid sequence information from at least a second pathogen strain;
   (ii) metadata identifying a second sampling location for said nucleic acid sequence information from said at least a second pathogen strain; and
   (ii) metadata identifying a second sampling time for said nucleic acid sequence information from said at least a second pathogen strain;
(d) applying, by said computer, said module for computing genetic distances to said nucleic acid sequence information from said pathogen strain and said at least a second pathogen strain to compute a genetic similarity between said pathogen strain and said at least a second pathogen strain;
(e) identifying said first pathogen strain and said at least a second pathogen strain as a same strain based at least in part on said genetic similarity.
Embodiment 2. The method of embodiment 1, further comprising (f) outputting a source location of said pathogen strain contamination at least in part based on said sampling time and sampling location metadata when said first pathogen strain and said at least a second pathogen strain are identified as a same strain.
Embodiment 3. The method of embodiment 1 or 2, wherein (a) further comprises detecting said pathogen in a sample among a plurality of samples, wherein said samples are taken from a plurality of physical locations at a plurality of different times.
Embodiment 4. The method of any one of embodiments 1-3, wherein (d) comprises determining a plurality of genetic distances between said nucleic acid sequence information from said pathogen and a plurality of nucleic acids from a plurality of suspect microbes from said second sample.
Embodiment 5. The method of embodiment 4, wherein genetic distances are computed between at least two orthologous or paralogous genes belonging to said first detected pathogen and plurality of suspect microbes.
Embodiment 6. The method of embodiment 5, wherein said genetic distance in is determined at least in part by calculating a number of unique nucleic acid base pairs between at least two orthologous or paralogous genes belonging to said first detected pathogen and plurality of suspect microbes.
Embodiment 7. The method of any one of embodiments 1-6, wherein (f) comprises ranking said samples contaminated with said pathogen according to said sampling time to identify an earliest contaminated sample representing the source of said contamination.
Embodiment 8. The method any one of embodiments 1-7, wherein said pathogen strain is a Listeria spp. Strain.
Embodiment 9. The method of any one of embodiments 1-8, further comprising receiving, at said computer, said nucleic acid sequence information from said pathogen strain, said nucleic acid sequence information from said at least a second pathogen strain, and said location and time metadata corresponding to said pathogen strain and said at least a second pathogen strain.
Embodiment 10. The method of embodiment 9, comprising receiving said nucleic acid sequence information from said pathogen strain, said nucleic acid sequence information from said at least a second pathogen strain, and said location and time metadata corresponding to said pathogen strain and said at least a second pathogen strain via a computer network.
Embodiment 11. The method of embodiment 10, wherein said computer network is the Internet, an internet and/or extranet, or an intranet and/or extranet that is in communication with the Internet.
Embodiment 12. The method of any one of embodiments 1-11, wherein (f) outputting said source location on a graphical map visible to an end-user.
Embodiment 13. The method of any one of embodiments 1-12, wherein (f) comprises transmission of said source location or said graphical map to an end user via a computer network.
Embodiment 14. The method of embodiment 13, wherein said computer network is the Internet, an internet and/or extranet, or an intranet and/or extranet that is in communication with the Internet.
Embodiment 15. A computer-implemented method of monitoring a pathogen strain, comprising,
(a) receiving, at a computer nucleic acid sequence information from said pathogen strain obtained from a first location at a first time;
(b) receiving, at said computer nucleic acid sequence information from at least a second pathogen strain obtained from at least a second location at at least a second time;
(c) determining, by said computer, a genetic similarity between said nucleic acid sequence information from said pathogen strain and said at least a second pathogen strain;
(e) identifying said first pathogen strain and said at least a second pathogen strain as a same strain based at least in part on said genetic similarity; and
(f) when said first pathogen strain and said at least a second pathogen strain are identified as a same strain, outputting a source location of said pathogen strain contamination at least in part based on metadata comprising said first location, said first time, said at least a second location, and said at least a second time.
Embodiment 16. Non-transitory computer-readable storage media encoded with a computer program including instructions executable by at least one processor to monitoring a pathogen strain comprising:
(a) a software module for receiving sequence information from a pathogen strain obtained from a first location at a first time and from at least a second pathogen strain obtained from at least a second location at at least a second time;
(b) a software module for determining a genetic similarity between said nucleic acid sequence information from said pathogen strain and said at least a second pathogen strain;
(c) a software module for identifying said first pathogen strain and said at least a second pathogen strain as a same pathogen based on said genetic similarity;
(d) a software module for outputting a source location of said pathogen strain contamination at least in part based on metadata comprising said first location, said first time, said at least a second location, and said at least a second time.
Embodiment 17. The storage media of embodiment 16, further comprising a software module for displaying a source location of said pathogen strain contamination on a graphical map.
Embodiment 18. The storage media of embodiment 17, wherein said software module further displays said first location and said at least a second location on said graphical map.
Embodiment 19. The storage media of embodiment 17 or 18, wherein said software module further displays said first time and said at least a second time along with said first location and said second location on said graphical map.
Embodiment 20. The storage media of embodiment 19, wherein said software module further displays one or more parameters not associated with sampling on said graphical map
Embodiment 21. The storage media of embodiment 20, wherein said one or more parameters not associated with sampling comprise employee movement patterns or residency at one or more of said locations on said graphical map, production quantities of a product at one or more locations on said graphical map, product flow between one or more locations on said graphical map, or reagent input flow between one or more locations on said graphical map.
Embodiment 22. The storage media of embodiment 16, comprising a module comprising a non-linear classification algorithm for computing a future sampling location for said pathogen strain based on a plurality of source locations calculated at different sampling times.
Embodiment 23. A method of monitoring a pathogen strain, comprising,
(a) identifying a location contaminated with said pathogen strain via detection of a first pathogen from a first sample;
(b) identifying a second location contaminated with said pathogen strain by computing a genetic similarity between said first detected pathogen and a second detected pathogen from a second sample;
(c) associating metadata comprising sampling time with said first and second location;
(d) identifying a source location of said pathogen strain contamination at least in part based on said metadata.
Embodiment 24. The method of embodiment 23, wherein (d) comprises identifying a source location of said pathogen strain contamination based on said sampling time and a genetic distance between said first detected pathogen and said second detected pathogen.
Embodiment 25. The method of embodiment 23 or 24, wherein (a) or (b) comprises detecting a pathogen in a sample among a plurality of samples, wherein said samples are taken from a plurality of physical locations at a plurality of different times.
Embodiment 26. The method of any one of embodiments 1-25, wherein said first or said second pathogen is identified by sequencing a nucleic acid derived from said first or said second pathogen.
Embodiment 27. The method of embodiment 25, wherein (b) comprises determining a plurality of genetic distances between a nucleic acid derived from said first pathogen and a nucleic acids derived from a plurality of suspect microbes from said second sample.
Embodiment 28. The method of embodiment 27, wherein genetic distances are computed between at least two orthologous or paralogous genes belonging to said first detected pathogen and plurality of suspect microbes.
Embodiment 29. The method of embodiment 28, wherein said genetic distance is determined at least in part by calculating a number of unique nucleic acid base pairs between at least two orthologous or paralogous genes belonging to said first detected pathogen and plurality of suspect microbes.
Embodiment 30. The method of any one of embodiments 1-29, wherein (d) comprises ranking said samples contaminated with said pathogen according to said sampling time to identify an earliest contaminated sample representing the source of said contamination.
Embodiment 31. The method of embodiment 1, wherein said pathogen strain is a Listeria spp. strain.
Embodiment 32. A method of monitoring the introduction of a new pathogen strain, comprising,
(a) detecting a pathogen in a sample among a plurality of samples, wherein said samples are taken from a plurality of physical locations;
(b) detecting a location contaminated with said pathogen among said plurality of physical locations via an association of said detection with said sample;
(c) determining, via a computer, a genetic distance between said detected pathogen and a most closely related microbe in said sample at said respective physical location;
(d) identifying said detected pathogen as transient or resident based on said genetic distance, thereby detecting said new introduced pathogen or the absence thereof.
Embodiment 33. The method of embodiment 32, further comprising
(e) when said detected pathogen is identified as transient, detecting a second location contaminated with said pathogen among said plurality of physical locations.
Embodiment 34. The method of embodiment 32, further comprising:
(f) associating metadata comprising sampling time of said samples with said first and second locations detected as contaminated; and
(g) identifying a first source of contamination among said locations detected as contaminated via said metadata.
Embodiment 35. A method of monitoring a pathogen strain, comprising
(a) detecting at least three locations contaminated with said pathogen strain among a plurality of physical locations via the detection of a pathogen from a plurality of samples from said plurality of locations;
(b) determining genetic distances among said detected pathogens at said contaminated locations; and
(c) clustering said detected pathogens from said contaminated locations according to said genetic distances to identify locations contaminated with at least a first strain and a second strain.
Embodiment 36. The method of embodiment 35, further comprising
(d) associating metadata comprising sampling time of said samples with said contaminated locations; and
(e) detecting a source of said first pathogen and a source of said second pathogen among said contaminated locations at least in part via said sampling time.
Embodiment 37. The method of any one of embodiments 32-36, wherein genetic distances are computed between at least two orthologous or paralogous genes of at least two pathogen strains or species.
Embodiment 38. The method of any one of embodiments 32-37, wherein said genetic distance in (b) is determined at least in part by calculating a number of unique nucleic acid base pairs between at least two orthologous or paralogous genes among said genes detected from said pathogen.
Embodiment 39. The method of embodiment 37 or 38, wherein said at least two orthologous or paralogous genes are selected from a 16S rRNA gene; an rps gene; a ribosomal protein L1p, L2p, L3p, L4p, L5p, L6p, L10p, L11p, L12p, L13p, L14p, L15p, L18p, L22p, L23p, L24p, L29p, L30p, S2p, S3p, S4p, S5p, S7p, S8p, S9p, S10p, S11p, S12p, S13p, S14p, S15p, S17p, S19p, and L7ae gene; a ribosomal protein L9p, L16p, L17p, L19p, L20p, L21p, L25p, L27p, L28p, L31p, L32p, L33p, L34p, L35p, L36p, S1p, S6p, S16p, S18p, S20p, S21p, S22p, and S31e gene; a ribosomal protein L10e, L13e, L14e, L15e, LXa/L18ae, L18e, L19e, L21e, L24e, L30e, L31e, L32e, L34e, L35ae, L37ae, L37e, L38e, L39e, L40e, L41e, L44e, S17e, S19e, S24e, S25e, S26e, S27ae, S27e, S28e, S30e, S3ae, S4e, S6e, S8e, L45a, L46a, and L47a gene.
Embodiment 40. The method of any one of embodiments 32-39, wherein said pathogen strain is a Listeria spp. strain.
Embodiment 41. The method of any one of embodiments 32-40, wherein said genetic distance in (c) is a Nei's standard distance, a Goldstein distance, a Reynolds/Weir/Cockerham's genetic distance, a Roger's distance, or a variant thereof.
Embodiment 42. The method of any one of embodiments 32-41, wherein (a) comprises generating a plurality of amplification products comprising at least one gene from said pathogen from said samples, wherein said amplification products are respectively spatially-addressable to said plurality of physical locations within said facility.
Embodiment 43. The method of embodiment 42, wherein (a) comprises performing a PCR reaction on nucleic acids derived from said samples utilizing oligonucleotide amplification primers containing unique sequences that are spatially addressable to said physical locations within said facility.
Embodiment 44. The method of any one of embodiments 32-43, wherein said facility is a food processing facility, a hospital, a pharmacy, a medical facility, or a clinical facility.
Embodiment 45. A method comprising:
(a) performing a PCR amplification reaction on a plurality of food or environmental samples from a plurality of physical locations within a facility, wherein said PCR reaction amplifies at least one gene from a Listeria spp. bacterium to generate a plurality of spatially-addressable amplification products containing said at least one gene;
(b) performing a sequencing reaction on said plurality of amplification products, wherein said sequencing reaction detects a gene characteristic to a particular Listeria spp. bacterium within said plurality of spatially-addressable amplification products; and
(d) associating, via a computer, the presence of said particular Listeria spp. bacterium with at least one of said plurality of physical locations within said facility via said spatially-addressable amplification product.
Embodiment 46. The method of embodiment 45, further comprising (e) outputting, via said computer, said at least one location contaminated with said particular Listeria spp. bacterium.
Embodiment 47. The method of embodiment 45 or 46, wherein said particular Listeria spp. bacterium is a pathogenic Listeria strain or species.
Embodiment 48. The method of any one of embodiments 1-44, wherein the pathogen strain includes a viral strain and a bacterial strain.
Embodiment 49. The method of embodiment 48, wherein the viral strain is a coronavirus strain.

While preferred embodiments of the present invention have been shown and described herein, such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A computer-implemented method of monitoring a pathogen strain, comprising,
(a) associating, at a computer:
(i) nucleic acid sequence information from said pathogen strain;
(ii) metadata identifying a first sampling location for said nucleic acid sequence information from said pathogen strain; and
(iii) metadata identifying a first sampling time for said nucleic acid sequence information from said pathogen strain;
(b) maintaining, in media accessible by said computer, a module for computing genetic distances between at least two nucleic acid sequences;
(c) associating, at said computer:
(i) nucleic acid sequence information from at least a second pathogen strain;
(ii) metadata identifying a second sampling location for said nucleic acid sequence information from said at least a second pathogen strain; and
(ii) metadata identifying a second sampling time for said nucleic acid sequence information from said at least a second pathogen strain;
(d) applying, by said computer, said module for computing genetic distances to said nucleic acid sequence information from said pathogen strain and said at least a second pathogen strain to compute a genetic similarity between said pathogen strain and said at least a second pathogen strain;
(e) identifying said first pathogen strain and said at least a second pathogen strain as a same strain based at least in part on said genetic similarity.

2. The method of claim 1, further comprising (f) outputting a source location of said pathogen strain contamination at least in part based on said sampling time and sampling location metadata when said first pathogen strain and said at least a second pathogen strain are identified as a same strain.

3. The method of claim 1, wherein (a) further comprises detecting said pathogen in a sample among a plurality of samples, wherein said samples are taken from a plurality of physical locations at a plurality of different times.

4. The method of claim 1, wherein (d) comprises determining a plurality of genetic distances between said nucleic acid sequence information from said pathogen and a plurality of nucleic acids from a plurality of suspect microbes from said second sample.

5. The method of claim 4, wherein genetic distances are computed between at least two orthologous or paralogous genes belonging to said first detected pathogen and plurality of suspect microbes.

6. The method of claim 5, wherein said genetic distance in is determined at least in part by calculating a number of unique nucleic acid base pairs between at least two orthologous or paralogous genes belonging to said first detected pathogen and plurality of suspect microbes.

7. The method of any one of claims 1-6, wherein (f) comprises ranking said samples contaminated with said pathogen according to said sampling time to identify an earliest contaminated sample representing the source of said contamination.

8. The method of claim 1, wherein said pathogen strain is a Listeria spp. Strain.

9. The method of claim 1, further comprising receiving, at said computer, said nucleic acid sequence information from said pathogen strain, said nucleic acid sequence information from said at least a second pathogen strain, and said location and time metadata corresponding to said pathogen strain and said at least a second pathogen strain.

10. The method of claim 9, comprising receiving said nucleic acid sequence information from said pathogen strain, said nucleic acid sequence information from said at least a second pathogen strain, and said location and time metadata corresponding to said pathogen strain and said at least a second pathogen strain via a computer network.

11. The method of claim 10, wherein said computer network is the Internet, an internet and/or extranet, or an intranet and/or extranet that is in communication with the Internet.

12. The method of claim 1, wherein (f) outputting said source location on a graphical map visible to an end-user.

13. The method of claim 1, wherein (f) comprises transmission of said source location or said graphical map to an end user via a computer network.

14. The method of claim 13, wherein said computer network is the Internet, an internet and/or extranet, or an intranet and/or extranet that is in communication with the Internet.

15. A computer-implemented method of monitoring a pathogen strain, comprising,
(a) receiving, at a computer nucleic acid sequence information from said pathogen strain obtained from a first location at a first time;
(b) receiving, at said computer nucleic acid sequence information from at least a second pathogen strain obtained from at least a second location at at least a second time;
(c) determining, by said computer, a genetic similarity between said nucleic acid sequence information from said pathogen strain and said at least a second pathogen strain;
(e) identifying said first pathogen strain and said at least a second pathogen strain as a same strain based at least in part on said genetic similarity; and
(f) when said first pathogen strain and said at least a second pathogen strain are identified as a same strain, outputting a source location of said pathogen strain contamination at least in part based on metadata comprising said first location, said first time, said at least a second location, and said at least a second time.

16. Non-transitory computer-readable storage media encoded with a computer program including instructions executable by at least one processor to monitoring a pathogen strain comprising:
(a) a software module for receiving sequence information from a pathogen strain obtained from a first location at a first time and from at least a second pathogen strain obtained from at least a second location at at least a second time;
(b) a software module for determining a genetic similarity between said nucleic acid sequence information from said pathogen strain and said at least a second pathogen strain;
(c) a software module for identifying said first pathogen strain and said at least a second pathogen strain as a same pathogen based on said genetic similarity;
(d) a software module for outputting a source location of said pathogen strain contamination at least in part based on metadata comprising said first location, said first time, said at least a second location, and said at least a second time.

17. The storage media of claim 16, further comprising a software module for displaying a source location of said pathogen strain contamination on a graphical map.

18. The storage media of claim 17, wherein said software module further displays said first location and said at least a second location on said graphical map.

19. The storage media of claim 17, wherein said software module further displays said first time and said at least a second time along with said first location and said second location on said graphical map.

20. The storage media of claim 19, wherein said software module further displays one or more parameters not associated with sampling on said graphical map

21. The storage media of claim 20, wherein said one or more parameters not associated with sampling comprise employee movement patterns or residency at one or more of said locations on said graphical map, production quantities of a product at one or more locations on said graphical map, product flow between one or more locations on said graphical map, or reagent input flow between one or more locations on said graphical map.

22. The storage media of claim 16, comprising a module comprising a non-linear classification algorithm for computing a future sampling location for said pathogen strain based on a plurality of source locations calculated at different sampling times.

23. A method of monitoring a pathogen strain, comprising,
(a) identifying a location contaminated with said pathogen strain via detection of a first pathogen from a first sample;
(b) identifying a second location contaminated with said pathogen strain by computing a genetic similarity between said first detected pathogen and a second detected pathogen from a second sample;
(c) associating metadata comprising sampling time with said first and second location;
(d) identifying a source location of said pathogen strain contamination at least in part based on said metadata.

24. The method of claim 23, wherein (d) comprises identifying a source location of said pathogen strain contamination based on said sampling time and a genetic distance between said first detected pathogen and said second detected pathogen.

25. The method of claim 23, wherein (a) or (b) comprises detecting a pathogen in a sample among a plurality of samples, wherein said samples are taken from a plurality of physical locations at a plurality of different times.

26. The method of claim 1, wherein said first or said second pathogen is identified by sequencing a nucleic acid derived from said first or said second pathogen.

27. The method of claim 25, wherein (b) comprises determining a plurality of genetic distances between a nucleic acid derived from said first pathogen and a nucleic acids derived from a plurality of suspect microbes from said second sample.

28. The method of claim 27, wherein genetic distances are computed between at least two orthologous or paralogous genes belonging to said first detected pathogen and plurality of suspect microbes.

29. The method of claim 28, wherein said genetic distance is determined at least in part by calculating a number of unique nucleic acid base pairs between at least two orthologous or paralogous genes belonging to said first detected pathogen and plurality of suspect microbes.

30. The method of claim 1, wherein (d) comprises ranking said samples contaminated with said pathogen according to said sampling time to identify an earliest contaminated sample representing the source of said contamination.
